(19) United States
(12) Patent Application Publication
(10) Pub. No.: US 2016/0301015 A1
(43) Pub. Date: Oct. 13, 2016

(54) LIGHT-EMITTING COMPOUNDS FOR LIGHT-EMITTING DEVICES

(71) Applicant: NITTO DENKO CORPORATION, Osaka (JP)

(72) Inventors: Shijun ZHENG, San Diego, CA (US); Adam BASIAGO, Temecula, CA (US)

(21) Appl. No.: 15/031,243

(22) PCT Filed: Oct. 30, 2014
(86) PCT No.: PCT/US2014/063207

§ 371 Date: Apr. 21, 2016

Related U.S. Application Data
(60) Provisional application No. 61/897,657, filed on Oct. 30, 2013.

(51) Int. Cl. H01L 51/00; C07D 209/86; C07D 209/80; C09K 11/06

(52) U.S. Cl. CPC H01L 51/0072; C09K 11/06; C07D 209/86; C07D 209/80; C09K 2211/1029; C09K 2211/1007; H01L 51/5016

(57) ABSTRACT

Compounds represented by Formula (1) are disclosed herein. Organic light-emitting elements and an organic light-emitting diode devices comprising these compounds are also disclosed.

### BACKGROUND

1. Field

Some embodiments relate to compounds for use in organic light-emitting diodes, such as for emissive materials.

2. Description of the Related Art

Organic light-emitting devices have been widely developed for flat panel displays, and are moving fast toward solid state lighting (SSL) applications. Organic Light-emitting Diodes (OLEDs) comprise a cathode, a hole transporting layer, an emissive layer, an electron transporting layer, and an anode. Light emitted from an OLED device may be the result of recombination of positive charges (holes) and negative charges (electrons) inside an organic (emissive) layer. The holes and electrons combine within a single molecule or a small cluster of molecules to generate excitons, which are molecules in an excited state, or groups of organic molecules bound together in an excited state. When the organic molecules release the required energy and return to their stable state, photons are generated. The organic compound or group of compounds which emit the photons are referred as an electro-fluorescent material or electro-phosphorescent material depending on the nature of the radiative process. Thus, the OLED emissive compounds may be selected for their ability to absorb primary radiation and emit radiation of a desired wavelength. For blue emitters, for example, emission within principle emission bands of 440 to 490 nm may be desirable.

For SSL applications, it may be helpful for a white OLED device to achieve greater than 1,500 lm brightness, a color rendering index (CRI) greater than 70, and an operating time greater than 100,000 hours at 1,000 cd/m². There are many approaches for generating white light from an OLED, but two common approaches are: direct combination of red, blue, and green light using either lateral patterning or vertical stacking of three emitters; and partial down conversion of blue light in combination with yellow phosphors. Both of these common approaches may be more effective if efficient chemical- and photo-stable dyes are employed. Thus, the development of emitter compounds with higher luminescence efficiency may be desirable to effectively reduce power consumption and generate emission of different colors.

### SUMMARY

Some embodiments include a compound represented by Formula 1:

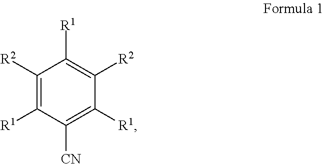

wherein each R¹ may be independently an electron acceptor moiety, such as an optionally substituted phenyl, optionally substituted pyridinyl, or an optionally substituted pyrimidinyl; and wherein each R² may be independently an electron donor moiety, such as an optionally substituted carbazolyl, an optionally substituted benzocarbazolyl, an optionally substituted phenoxazinyl, an optionally substituted phenothiazinyl, or an optionally substituted dihydrophenazinyl.

Some embodiments include optionally substituted (4′r,6′r)-5′-(4-cyanophenyl)-4′,6′-bis(3,6-diphenyl-9H-carbazol-9-yl)-[1,1′:3′,1″-terphenyl]-2′,4,4″-tricarbonitrile (EC-1); optionally substituted (4′R,6′R)-4′,6′-bis(7H-benzo[c]carbazol-7-yl)-5′-(4-cyanophenyl)-[1,1′:3′,1″-terphenyl]-2′,4,4″-tricarbonitrile (EC-2); optionally substituted (4′R,6′R)-4′,6′-bis(5H-benzo[b]carbazol-5-yl)-5′-(4-cyanophenyl)-[1,1′:3′,1″-terphenyl]-2′,4,4″-tricarbonitrile (EC-3); optionally substituted (4′R,6′R)-4′,6′-bis(7H-benzo[c]carbazol-7-yl)-4,4″-bis(trifluoromethyl)-5′-(4-(trifluoromethyl)phenyl)-[1,1′:3′,1″-terphenyl]-2′-carbonitrile (EC-4); optionally substituted (4′R,6′R)-4′,6′-bis(5H-benzo[b]carbazol-5-yl)-4,4″-bis(trifluoromethyl)-5′-(4-(trifluoromethyl)phenyl)-[1,1′:3′,1″-terphenyl]-2′-carbonitrile (EC-5); optionally substituted (S)-5′-(4-cyanophenyl)-4′,6′-bis(3-(diphenylamino)-9H-carbazol-9-yl)-[1,1′:3′,1″-terphenyl]-2′,4,4″-tricarbonitrile (EC-6); optionally substituted (S)-3,5-bis(6-cyanopyridin-3-yl)-2,6-bis(3-(diphenylamino)-9H-carbazol-9-yl)-[1,1′-biphenyl]-4,4′-dicarbonitrile (EC-7); optionally substituted (4′r,6′R)-4′,6′-bis(9′H-[9,3′:6′,9″-tercarbazol]-9′-yl)-5′-(4-cyanophenyl)-[1,1′:3′,1″-terphenyl]-2′,4,4″-tricarbonitrile (EC-8); optionally substituted (S)-4′,6′-bis(3-(10H-phenoxazin-10-yl)-9H-carbazol-9-yl)-5′-(4-cyanophenyl)-[1,1′:3′,1″-terphenyl]-2′,4,4″-tricarbonitrile (EC-9); optionally substituted (S)-5,5′,5″-(2,4-bis(3-(10H-phenoxazin-10-yl)-9H-carbazol-9-yl)-6-cyanobenzene-1,3,5-triyl)tripicolinonitrile (EC-10); or optionally substituted (s)-4′,6′-bis(3,6-bis(diphenylamino)-9H-carbazol-9-yl)-5′-(4-cyanophenyl)-[1,1′:3′,1″-terphenyl]-2′,4,4″-tricarbonitrile (EC-11).

Some embodiments include an organic light-emitting diode device comprising a cathode; an anode; a light-emitting layer disposed between and electrically connected to the anode and the cathode; a hole-transport layer between the anode and the light-emitting layer; and an electron-transport layer between the cathode and the light-emitting layer; wherein the light-emitting layer, the hole-transport layer, or the electron-transport layer comprise a host compound described herein.

Some embodiments may include an organic light-emitting diode device comprising: a cathode; an anode; and a light-emitting layer disposed between and electrically connected to the anode and the cathode; wherein the light-emitting layer comprises a host compound described herein.

These and other embodiments are described in greater detail below.

### BRIEF DESCRIPTION OF THE DRAWINGS

FIG. 1 is a schematic of a device incorporating an embodiment of a compound described herein.

### DETAILED DESCRIPTION

By employing a newly designed molecular structure, an example shown below, a series of emissive materials are described that can be used in OLED device applications.

Unless otherwise indicated, when a compound or chemical structural feature such as phenyl, pyridinyl, pyrimidyl, carbazolyl, benzocarbazolyl, phenoxazinyl, phenothiazinyl, or dihydrophenazinyl is referred to as being “optionally substituted,” it includes a feature that has no substituents (i.e. unsubstituted), or a feature that is substituted, meaning that the feature has one or more substituents. The term “substituent” has the broadest meaning known to one of ordinary skill in the art, and includes a moiety that replaces one or more hydrogen atoms in a parent compound or structural feature. The term “replaces” is merely used herein for convenience, and does not require that the compound be formed by replacing one atom with another. In some embodiments, a substituent may be any ordinary organic moiety known in the art, which may have a molecular weight (e.g. the sum of the atomic masses of the atoms of the substituent) of 15 Da to 50 Da, 15 Da to 100 Da, 15 Da to 150 Da, 15 Da to 200 Da, 15 Da to 300 Da, or 15 Da to 500 Da. In some embodiments, a substituent comprises, or consists of: 0-30, 0-20, 0-10, or 0-5 carbon atoms; and 0-30, 0-20, 0-10, or 0-5 heteroatoms, wherein each heteroatom may independently be: N, O, P, S, Si, F, Cl, Br, or I; provided that the substituent includes one C, N, O, P, S, Si, F, Cl, Br, or I atom. In some embodiments, substituents can consist of 2 to 5 chemical elements, wherein the chemical elements are independently C, H, O, N, P, S, Si, F, Cl, or Br. In some embodiments, a substituent is optionally substituted alkyl, —O-alkyl (e.g. —OCH₃, —OC₂H₅, —OC₃H₇, —OC₄H₉, etc.), —S-alkyl (e.g. —SCH₃, —SC₂H₅, —SC₃H₇, —SC₄H₉, etc.), —NR′R″, —OH, —SH, —CN, —CF₃, —NO₂, perfluoroalkyl, optionally substituted aryl, optionally substituted heteroaryl, optionally substituted amine or a halogen, wherein R′ and R″ are independently H or optionally substituted alkyl. Wherever a substituent is described as “optionally substituted,” that substituent can be substituted with the above substituents.

As used herein, the term “alkyl” has the broadest meaning generally understood in the art, and may include a moiety composed of carbon and hydrogen containing no double or triple bonds. Alkyl may be linear alkyl, branched alkyl, cycloalkyl, or a combination thereof, and in some embodiments, may contain from one to thirty-five carbon atoms. In some embodiments, alkyl may include C₁₋₁₀ linear alkyl, such as methyl (—CH₃), ethyl (—CH₂CH₃), n-propyl (—CH₂CH₂CH₃), n-butyl (—CH₂CH₂CH₂CH₃), n-pentyl (—CH₂CH₂CH₂CH₂CH₃), n-hexyl (—CH₂CH₂CH₂CH₂CH₂CH₃), etc.; C₃₋₁₀ branched alkyl, such as C₃H₇ (e.g. iso-propyl), C₄H₉ (e.g. branched butyl isomers), C₅H₁₁ (e.g. branched pentyl isomers), C₆H₁₃ (e.g. branched hexyl isomers), C₇H₁₅ (e.g. branched heptyl isomers), etc.; C₃₋₁₀ cycloalkyl, such as C₃H₅ (e.g. cyclopropyl), C₄H₇ (e.g. cyclobutyl isomers such as cyclobutyl, methylcyclopropyl, etc.), C₅H₉ (e.g. cyclopentyl isomers such as cyclopentyl, methylcyclobutyl, dimethylcyclopropyl, etc.) C₆H₁₁ (e.g. cyclohexyl isomers), C₇H₁₃ (e.g. cycloheptyl isomers), etc.; and the like.

The term “phenyl” refers to:

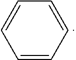

The term “pyridinyl” refers to:

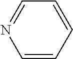

and includes

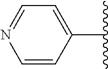 [SMILES: *c1ccncc1]

(pyridin-4-yl),

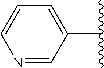 [SMILES: *c1cccnc1]

(pyridin-3-yl), and

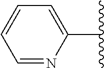 [SMILES: *c1ccccn1]

(pyridin-2-yl).

The term “pyrimidinyl” refers to:

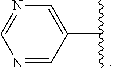 [SMILES: *c1cncnc1]

The term “carbazolyl” refers to the ring system:

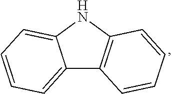 [SMILES: c1ccc2c(c1)[nH]c1ccccc12]

which includes, but is not limited to

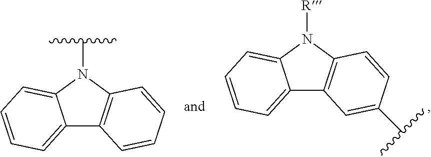

wherein R″′ can be H, C₁-C₃ alkyl, C₁-C₃ perfluoroalkyl, optionally substituted aryl, e.g., an optionally substituted phenyl, optionally substituted carbazolyl, optionally substituted amine, optionally substituted phenoxazinyl, optionally substituted phenothiazinyl, and optionally substituted dihydrophenazinyl.

The term “benzocarbazolyl” refers to a ring system which includes, but is not limited to

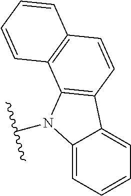 [SMILES: *n1c2ccccc2c2ccc3ccccc3c21]

(benzo[a]carbazolyl),

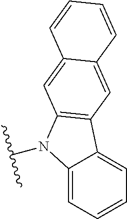 [SMILES: *n1c2ccccc2c2cc3ccccc3cc21]

(benzo[b]carbazolyl), and/or

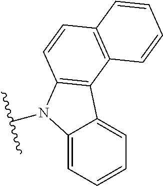 [SMILES: *n1c2ccccc2c2c3ccccc3ccc21]

(benzo[c]carbazolyl).

The term “phenoxazinyl” refers to a ring system that includes

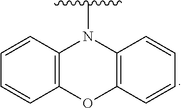 [SMILES: *N1c2ccccc2Oc2ccccc21]

The term “phenothiazinyl” refers to the ring system that includes

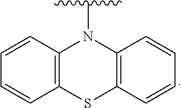 [SMILES: *N1c2ccccc2Sc2ccccc21]

The term “dihydrophenazinyl” refers to the ring system that includes:

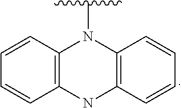 [SMILES: *N1c2ccccc2Nc2ccccc21]

Some embodiments include an emissive compound for use in emissive elements of organic light-emitting devices, wherein the compound is represented by Formula 1:

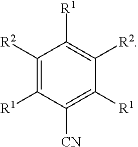

With respect to any relevant structural representation, such as Formula 1, each R¹ can independently be a moiety that can be an electron acceptor. In some embodiments, R¹ can be aryl having a CN substituent or heteroaryl having a CN substituent. In some embodiments, R¹ can be aryl having a CF₃ substituent or heteroaryl having a CF₃ substituent. In some embodiments, R¹ can be an optionally substituted phenyl, an optionally substituted pyridinyl, and/or an optionally substituted pyrimidinyl. In some embodiments, the phenyl, pyridinyl, or pyrimidyl has a CN substituent. In some embodiments, the phenyl, pyridinyl, or pyrimidyl has a CF₃ substituent. In some embodiments, each R¹ can independently be any of:

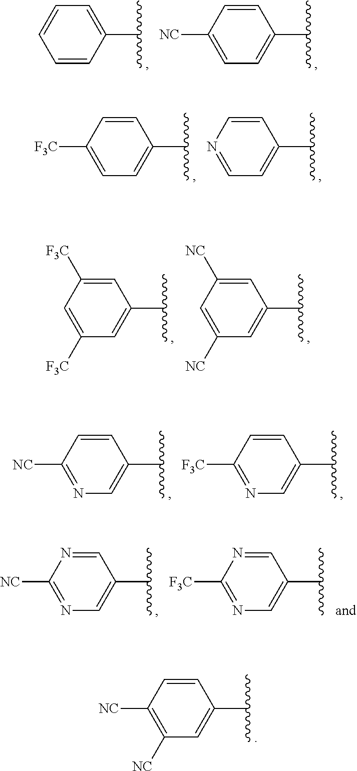

Some embodiments include a compound represented by Formula 1A:

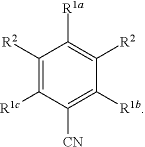

In some embodiments, R^{1a} can be

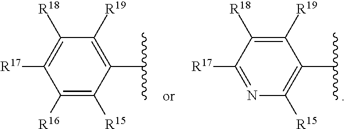

With respect to any relevant structural representation, such as Formula 1A, each R^{1a} can independently be a moiety that can be an electron acceptor. In some embodiments, R^{1a} can be aryl having a CN substituent or heteroaryl having a CN substituent. In some embodiments, R^{1a} can be aryl having a CF₃ substituent or heteroaryl having a CF₃ substituent. In some embodiments, R^{1a} can be an optionally substituted phenyl, an optionally substituted pyridinyl, and/or an optionally substituted pyrimidinyl. In some embodiments, the phenyl, pyridinyl, or pyrimidyl has a CN substituent. In some embodiments, the phenyl, pyridinyl, or pyrimidyl has a CF₃ substituent. In some embodiments, each R^{1a} can independently be any of:

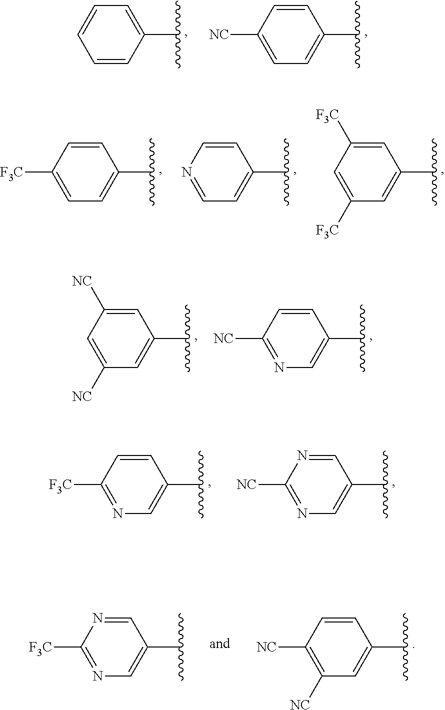

In some embodiments, R^{1b} can be

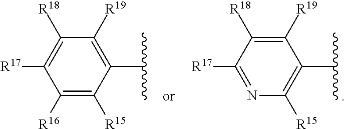

With respect to any relevant structural representation, such as Formula 1A, R^{1b} can be a moiety that can be an electron acceptor. In some embodiments, R^{1b} can be aryl having a CN substituent or heteroaryl having a CN substituent. In some embodiments, R^{1b} can be aryl having a CF₃ substituent or heteroaryl having a CF₃ substituent. In some embodiments, R^{1b} can be an optionally substituted phenyl, an optionally substituted pyridinyl, and/or an optionally substituted pyrimidinyl. In some embodiments, the phenyl, pyridinyl, or pyrimidyl has a CN substituent. In some embodiments, the phenyl, pyridinyl, or pyrimidyl has a CF₃ substituent. In some embodiments, each R^{1b} can independently be any of optionally substituted:

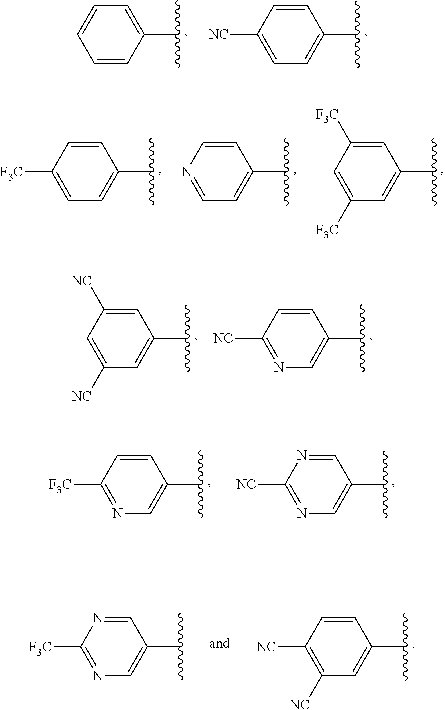

In some embodiments, R^{1c} can be

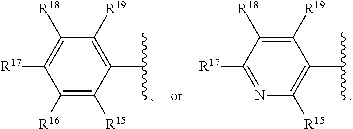

With respect to any relevant structural representation, such as Formula 1A, R^{1c} can be a moiety that can be an electron acceptor. In some embodiments, R^{1c} can be aryl having a CN substituent or heteroaryl having a CN substituent. In some embodiments, R^{1c} can be aryl having a CF₃ substituent or heteroaryl having a CF₃ substituent. In some embodiments, R^{1c} can be an optionally substituted phenyl, an optionally substituted pyridinyl, and/or an optionally substituted pyrimidinyl. In some embodiments, the phenyl, pyridinyl, or pyrimidyl has a CN substituent. In some embodiments, the phenyl, pyridinyl, or pyrimidyl has a CF₃ substituent. In some embodiments, each R^{1c} can independently be any of optionally substituted:

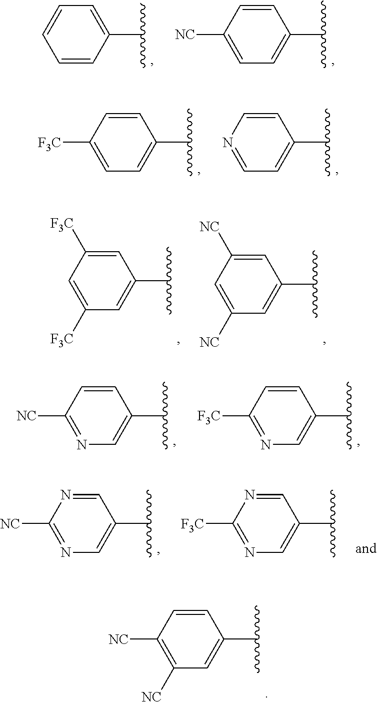

With respect to any relevant structural representation, such as Formula 1 or Formula 1A, each R² can independently be a substituent that can be an electron donor. In some embodiments, each R² independently can be any one of an optionally substituted carbazolyl; an optionally substituted benzocarbazolyl, such as optionally substituted benzo[a]carbazolyl, optionally substituted benzo[b]carbazolyl, or optionally substituted benzo[c]carbazolyl; optionally substituted phenoxazinyl, optionally substituted phenothiazinyl; and/or optionally substituted dihydrophenazinyl. In some embodiments, each R² independently can be any one of

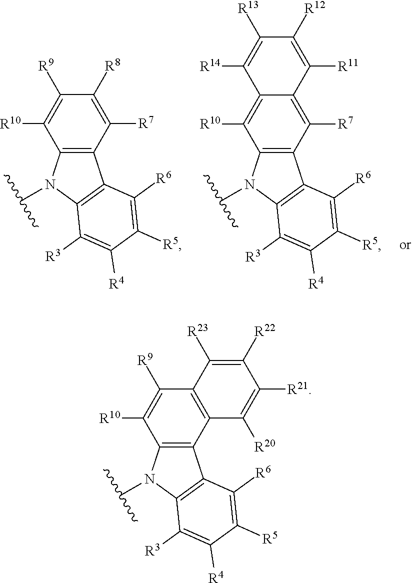

In some embodiments, each R² can independently be any of optionally substituted:

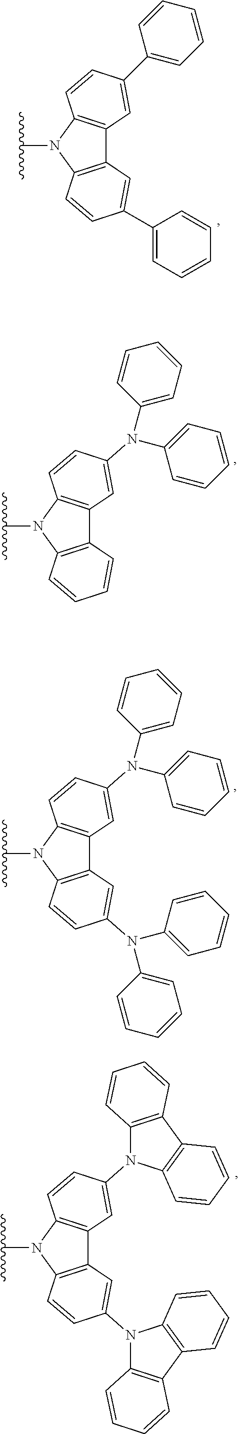

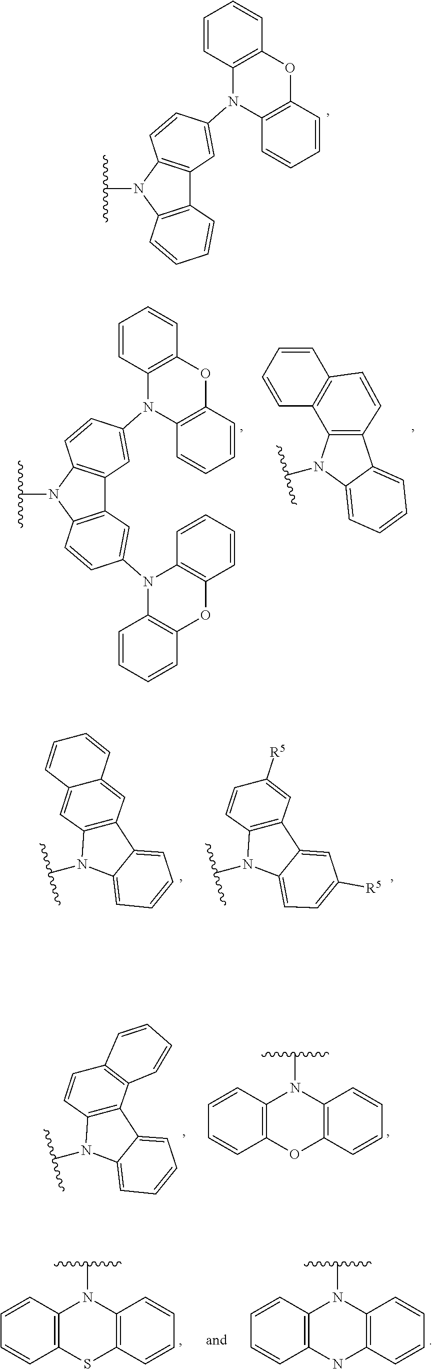

Some embodiments include a compound represented by Formula 2:

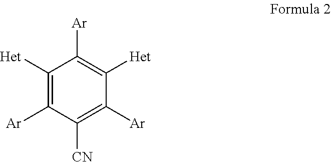

wherein each Ar is independently optionally substituted aryl; and each Het is independently optionally substituted heteroaryl.

The term “aryl” as used herein refers to an aromatic ring or ring system. Exemplary non-limiting aryl groups are phenyl, naphthyl, etc. “C_{x-y} aryl” refers to aryl where the ring or ring system has x-y carbon atoms. For example, “C₆₋₁₀ aryl” has a ring or ring system with 6 to 10 carbon atoms. The indicated number of carbon atoms for the ring or ring system does not include or limit the number of carbon atoms in any substituents attached to the ring or ring system. Examples include, but are not limited to, optionally substituted phenyl, optionally substituted naphthyl, optionally substituted anthracenyl, optionally substituted p-interphenylene, optionally substituted 1,4-internaphthylene, and optionally substituted 9,10-interanthracenylene. These are shown below in their unsubstituted forms. However, any carbon not attached to the remainder of the molecule may optionally have a substituent.

In some embodiments, any Ar can be optionally substituted:

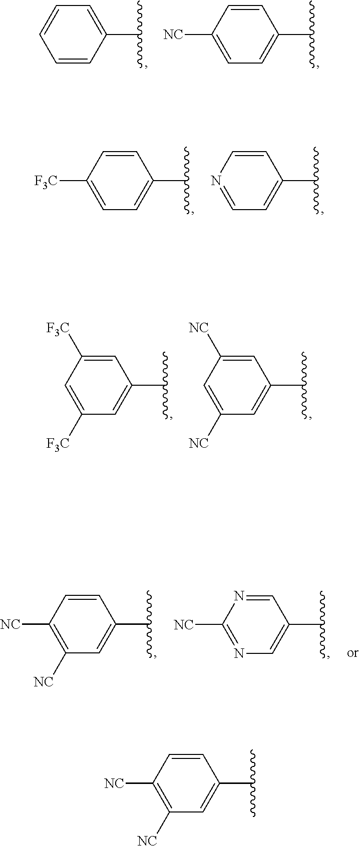

The term “heteroaryl” refers to “aryl” which has one or more heteroatoms in the ring or ring system. “C_{x-y} heteroaryl” refers to heteroaryl where the ring or ring system has x-y carbon atoms. The indicated number of carbon atoms for the ring or ring system does not include or limit the number of carbon atoms in any substituents attached to the ring or ring system. Examples of “heteroaryl” may include, but are not limited to, carbazolyl, benzocarbazolyl, pyridinyl, pyrimidinyl, furyl, thienyl, oxazolyl, thiazolyl, imidazolyl, indolyl, quinolinyl, benzofuranyl, benzothienyl, benzooxazolyl, benzothiazolyl, benzoimidazolyl, phenoxazinyl, phenothiazinyl, dihydrophenazinyl, etc. In some embodiments, the “heteroaryl” can be an optionally substituted pyridinyl. In some embodiments, the “heteroaryl” can be an optionally substituted pyrimidinyl. In some embodiments, the “heteroaryl” can be an optionally substituted carbazolyl. In some embodiments, the “heteroaryl” can be an optionally substituted benzocarbazolyl. In some embodiments, the “heteroaryl” can be an optionally substituted phenoxazinyl. In some embodiments, the “heteroaryl” can be an optionally substituted phenothiazinyl. In some embodiments, the “heteroaryl” can be an optionally substituted dihydrophenazinyl.

In some embodiments, any Het can be optionally substituted:

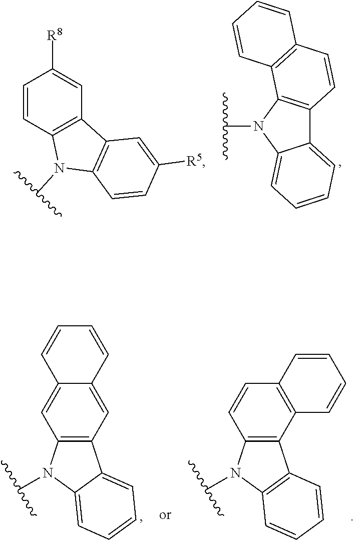

In some embodiments, each R⁸ and R⁵ can each independently be an optionally substituted phenyl, C₁-C₃ alkyl, e.g., methyl, ethyl, t-butyl; optionally substituted carbazolyl, optionally substituted amine, optionally substituted phenoxazinyl, optionally substituted phenothiazinyl, optionally substituted dihydrophenazinyl, and hydrogen. In some embodiments, the C₁-C₃ alkyl can be methyl and/or tert-butyl.

In some embodiments, R⁸ can be:

Hydrogen,

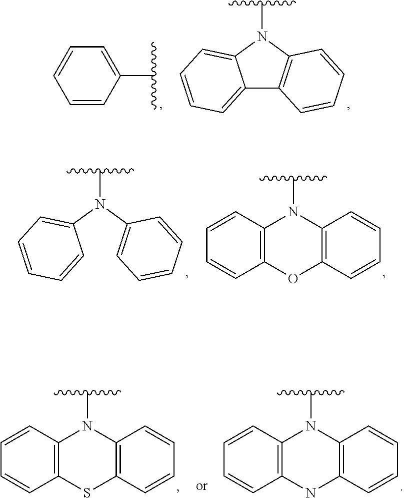

In some embodiments, R⁵ can be:

### Hydrogen,

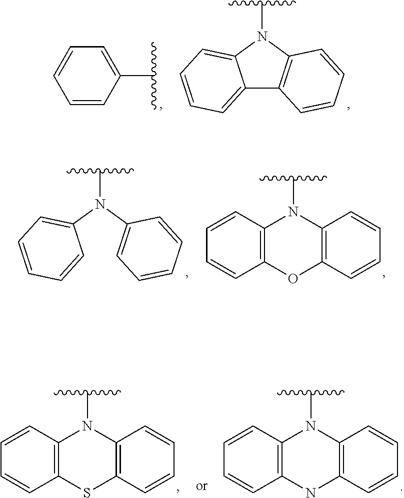

With respect to any relevant structural representation, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R²³ may independently be H or any substituent, such as a substituent having from 0 to 6 carbon atoms and from 0 to 5 heteroatoms, wherein each heteroatom is independently: O, N, P, S, F, Cl, Br, or I; and/or having a molecular weight of 15 g/mol to 300 g/mol, or 15 g/mol to 150 g/mol. In some embodiments, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R²³ are independently R^{A}, F, Cl, CN, OR^{A}, CF₃, NO₂, NR^{A}R^{B}, COR^{A}, CO₂R^{A}, OCOR^{A}, NR^{A}COR^{B}, CONR^{A}R^{B}, etc. In some embodiments, R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R²³ are independently H; F; CI; CN; CF₃; OH; NH₂; C₁₋₆ alkyl, such as methyl, ethyl, propyl isomers (e.g. n-propyl and isopropyl), cyclopropyl, butyl isomers, cyclobutyl isomers (e.g. cyclobutyl and methylcyclopropyl), pentyl isomers, cyclopentyl isomers, hexyl isomers, cyclohexyl isomers, etc.; or C₁₋₆ alkoxy, such as —O-methyl, —O-ethyl, isomers of —O-propyl, —O-cyclopropyl, isomers of —O-butyl, isomers of —O-cyclobutyl, isomers of —O-pentyl, isomers of —O-cyclopentyl, isomers of —O-hexyl, isomers of —O-cyclohexyl, optionally substituted enyl, optionally substituted carbazolyl, optionally substituted amine, etc.

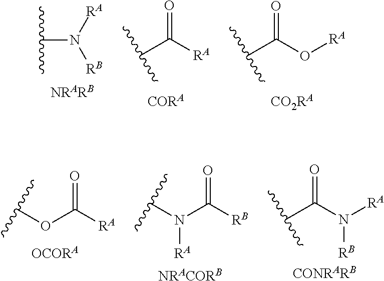

With respect to any relevant structural representation, each R^{A} may independently be H, or C₁₋₁₂ alkyl, including: linear or branched alkyl having a formula CₐHₐ₊₁, or cycloalkyl having a formula CₐHₐ₋₁, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₇H₁₅, C₈H₁₇, C₉H₁₉, C₁₀H₂₁, etc., or cycloalkyl of a formula: C₃H₅, C₄H₇, C₅H₉, C₆H₁₁, C₇H₁₃, C₈H₁₅, C₉H₁₇, C₁₀H₁₉, etc. In some embodiments, R^{A} may be H or C₁₋₆ alkyl. In some embodiments, R^{A} may be H or C₁₋₃ alkyl. In some embodiments, R^{A} may be H or CH₃. In some embodiments, R^{A} may be H.

With respect to any relevant structural representation, each R^{B} may independently be H, or C₁₋₁₂ alkyl, including: linear or branched alkyl having a formula CₐHₐ₊₁, or cycloalkyl having a formula CₐHₐ₋₁, wherein a is 1, 2, 3, 4, 5, 6, 7, 8, 9, 10, 11, or 12, such as linear or branched alkyl of a formula: CH₃, C₂H₅, C₃H₇, C₄H₉, C₅H₁₁, C₆H₁₃, C₈H₁₇, C₇H₁₅, C₉H₁₉, C₁₀H₂₁, etc., or cycloalkyl of a formula: C₃H₅, C₄H₇, C₅H₉, C₆H₁₁, C₇H₁₃, C₈H₁₅, C₉H₁₇, C₁₀H₁₉, etc. In some embodiments, R^{B} may be H or C₁₋₃ alkyl. In some embodiments, R^{B} may be H or CH₃. In some embodiments, R^{B} may be H.

With respect to any relevant structural representation, in some embodiments, R³ is H, F, Cl, or CH₃. In some embodiments, R³ is H. Additionally, for any embodiments recited in this paragraph, R⁴, R⁵, and R⁶ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted phenyl, optionally substituted carbazolyl, optionally substituted amine, or C₁₋₆ alkoxy.

With respect to any relevant structural representation, in some embodiments, R⁴ is H, F, Cl, or CH₃. In some embodiments, R⁴ is H. Additionally, for any embodiments recited in this paragraph, R³, R⁵, and R⁶ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted phenyl, optionally substituted carbazolyl, optionally substituted amine, or C₁₋₆ alkoxy.

With respect to any relevant structural representation, in some embodiments, R⁵ is H, F, Cl, or CH₃. In some embodiments, R⁵ is H, optionally substituted phenyl, optionally substituted carbazolyl, optionally substituted diphenylamino, or optionally substituted phenoxazinyl. In some embodiments, R⁵ is H. In some embodiments, R⁵ is optionally substituted phenyl. In some embodiments, R⁵ is optionally substituted carbazolyl. In some embodiments, R⁵ is optionally substituted diphenylamino. In some embodiments, R⁵ is optionally substituted phenoxazinyl. Additionally, for any embodiments recited in this paragraph, R³, R⁴, and R⁶ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted phenyl, optionally substituted carbazolyl, optionally substituted amine, or C₁₋₆ alkoxy.

With respect to any relevant structural representation, in some embodiments, R⁶ is H, F, Cl, or CH₃. In some embodiments, R⁶ is H. Additionally, for any embodiments recited in this paragraph, R³, R⁴, and R⁵ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R⁷ is H, F, Cl, or CH₃. In some embodiments, R⁷ is H. Additionally, for any embodiments recited in this paragraph, R⁸, R⁹, and R¹⁰ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R⁸ is H, F, Cl, or CH₃. In some embodiments, R⁸ is H, optionally substituted phenyl, optionally substituted carbazolyl, optionally substituted diphenylamino, or optionally substituted phenoxazinyl. In some embodiments, R⁸ is H. In some embodiments, R⁸ is optionally substituted phenyl. In some embodiments, R⁸ is optionally substituted carbazolyl. In some embodiments, R⁸ is optionally substituted diphenylamino. In some embodiments, R⁸ is optionally substituted phenoxazinyl. Additionally, for any embodiments recited in this paragraph, R⁷, R⁹, and R¹⁰ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted phenyl, optionally substituted carbazolyl, optionally substituted amine, or C₁₋₆ alkoxy.

With respect to any relevant structural representation, in some embodiments, R⁹ is H, F, Cl, or CH₃. In some embodiments, R⁹ is H. Additionally, for any embodiments recited in this paragraph, R⁷, R⁸, and R¹⁰ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹⁰ is H, F, Cl, or CH₃. In some embodiments, R¹⁰ is H. Additionally, for any embodiments recited in this paragraph, R¹¹, R¹², R¹³ and R¹⁴ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹¹ is H, F, Cl, or CH₃. In some embodiments, R¹¹ is H. Additionally, for any embodiments recited in this paragraph, R¹⁰, R¹², R¹³ and R¹⁴ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹² is H, F, Cl, or CH₃. In some embodiments, R¹² is H. Additionally, for any embodiments recited in this paragraph, R¹⁰, R¹¹, R¹³ and R¹⁴ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹³ is H, F, Cl, or CH₃. In some embodiments, R¹³ is H. Additionally, for any embodiments recited in this paragraph, R¹⁰, R¹¹, R¹² and R¹⁴ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹⁴ is H, F, Cl, or CH₃. In some embodiments, R¹⁴ is H. Additionally, for any embodiments recited in this paragraph, R¹⁰, R¹¹, R¹² and R¹³ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹⁵ is H, F, Cl, CN, CF₃, or CH₃. In some embodiments, R¹⁵ is H. Additionally, for any embodiments recited in this paragraph, R¹⁶, R¹⁷, R¹⁸ and R¹⁹ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹⁶ is H, F, Cl, CN, CF₃, or CH₃. In some embodiments, R¹⁶ is H. Additionally, for any embodiments recited in this paragraph, R¹⁵, R¹⁷, R¹⁸ and R¹⁹ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹⁷ is H, F, Cl, CN, CF₃, or CH₃. In some embodiments, R¹⁷ is H. In some embodiments, R¹⁷ is CN. In some embodiments, R¹⁷ is CF₃. Additionally, for any embodiments recited in this paragraph, R¹⁵, R¹⁶, R¹⁸ and R¹⁹ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹⁸ is H, F, Cl, CN, CF₃, or CH₃. In some embodiments, R¹⁸ is H. Additionally, for any embodiments recited in this paragraph, R¹⁵, R¹⁶, R¹⁷ and R¹⁹ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R¹⁹ is H, F, Cl, CN, CF₃, or CH₃. In some embodiments, R¹⁹ is H. Additionally, for any embodiments recited in this paragraph, R¹⁵, R¹⁶, R¹⁷ and R¹⁸ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R² is H, F, Cl, CN, CF₃, or CH₃. In some embodiments, R² is H. Additionally, for any embodiments recited in this paragraph, R²¹, R²², R²³ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R²¹ is H, F, Cl, CN, CF₃, or CH₃. In some embodiments, R²¹ is H. Additionally, for any embodiments recited in this paragraph, R²⁰, R²², and R²³ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R²² is H, F, Cl, CN, CF₃, or CH₃. In some embodiments, R²² is H. Additionally, for any embodiments recited in this paragraph, R²⁰, R²¹, and R²³ can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

With respect to any relevant structural representation, in some embodiments, R²³ is H, F, Cl, CN, CF₃, or CH₃. In some embodiments, R²³ is H. Additionally, for any embodiments recited in this paragraph, R²⁰, R²¹, and R²² can independently be: R^{A}, F, Cl, CN, OR^{A}, CF₃, NO₂, NR^{A}R^{B}, COR^{A}, CO₂R^{A}, OCOR^{A}, NR^{A}COR^{B}, or CONR^{A}R^{B}; or H, F, Cl, CN, CF₃, OH, NH₂, C₁₋₆ alkyl, optionally substituted carbazolyl, optionally substituted amine, or C₁₋₆ alkoxy.

In some embodiments, the compound can be:

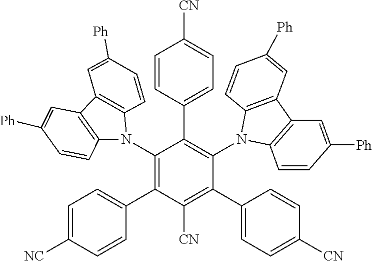 [SMILES: N#Cc1ccc(-c2c(C#N)c(-c3ccc(C#N)cc3)c(-n3c4ccc(-c5ccccc5)cc4c4cc(-c5ccccc5)ccc43)c(-c3ccc(C#N)cc3)c2-n2c3ccc(-c4ccccc4)cc3c3cc(-c4ccccc4)ccc32)cc1]

(4′r,6′r)-5′-(4-cyanophenyl)-4′,6′-bis(3,6-diphenyl-9H-carbazol-9-yl)-[1,1′: 3′,1″-terphenyl]-2′,4,4″-tricarbonitrile (EC-1)

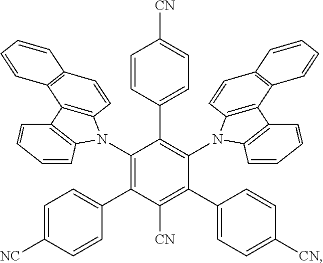 [SMILES: N#Cc1ccc(-c2c(C#N)c(-c3ccc(C#N)cc3)c(-n3c4ccccc4c4c5ccccc5ccc43)c(-c3ccc(C#N)cc3)c2-n2c3ccccc3c3c4ccccc4ccc32)cc1]

(4′R,6′R)-4′,6′-bis(7H-benzo[c]carbazol-7-yl)-5′-(4-cyanophenyl)-[1,1′:3′,1″-terphenyl]-2′,4,4″-tricarbonitrile (EC-2);

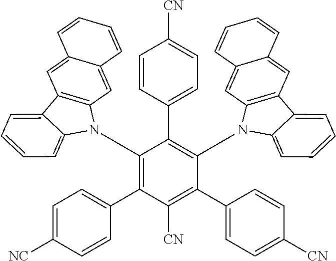 [SMILES: N#Cc1ccc(-c2c(C#N)c(-c3ccc(C#N)cc3)c(-n3c4ccccc4c4cc5ccccc5cc43)c(-c3ccc(C#N)cc3)c2-n2c3ccccc3c3cc4ccccc4cc32)cc1]

(4′R,6′R)-4′,6′-bis(5H-benzo[b]carbazol-5-yl)-5′-(4-cyanophenyl)-[1,1′:3′,1″-terphenyl]-2′,4,4″-tricarbonitrile (EC-3);

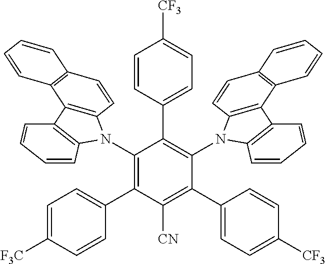 [SMILES: N#Cc1c(-c2ccc(C(F)(F)F)cc2)c(-n2c3ccccc3c3c4ccccc4ccc32)c(-c2ccc(C(F)(F)F)cc2)c(-n2c3ccccc3c3c4ccccc4ccc32)c1-c1ccc(C(F)(F)F)cc1]

(4′R,6′R)-4′,6′-bis(7H-benzo[c]carbazol-7-yl)-4,4″-bis(trifluoromethyl)-5′-(4-(trifluoromethyl)phenyl)-[1,1′:3′,1″-terphenyl]-2′-carbonitrile (EC-4);

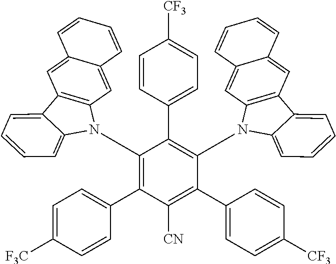 [SMILES: N#Cc1c(-c2ccc(C(F)(F)F)cc2)c(-n2c3ccccc3c3cc4ccccc4cc32)c(-c2ccc(C(F)(F)F)cc2)c(-n2c3ccccc3c3cc4ccccc4cc32)c1-c1ccc(C(F)(F)F)cc1]

(4′R,6′R)-4′,6′-bis(5H-benzo[b]carbazol-5-yl)-4,4″-bis(trifluoromethyl)-5′-(4-(trifluoromethyl)phenyl)[1,1′:3′,1″-terphenyl]-2′-carbonitrile (EC-5);

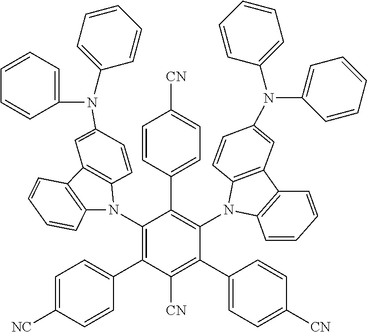 [SMILES: N#Cc1ccc(-c2c(C#N)c(-c3ccc(C#N)cc3)c(-n3c4ccccc4c4cc(N(c5ccccc5)c5ccccc5)ccc43)c(-c3ccc(C#N)cc3)c2-n2c3ccccc3c3cc(N(c4ccccc4)c4ccccc4)ccc32)cc1]

(S)-5′-(4-cyanophenyl)-4′,6′-bis(3-(diphenylamino)-9H-carbazol-9-yl)[1,1′:3′,1″-terphenyl]-2′,4,4″-tricarbonitrile (EC-6);

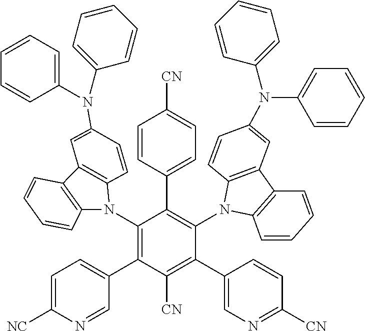 [SMILES: N#Cc1ccc(-c2c(-n3c4ccccc4c4cc(N(c5ccccc5)c5ccccc5)ccc43)c(-c3ccc(C#N)nc3)c(C#N)c(-c3ccc(C#N)nc3)c2-n2c3ccccc3c3cc(N(c4ccccc4)c4ccccc4)ccc32)cc1]

(S)-3,5-bis(6-cyanopyridin-3-yl)-2,6-bis(3-(diphenylamino)-9H-carbazol-9-yl)-[1,1′-biphenyl]-4,4′-dicarbonitrile (EC-7);

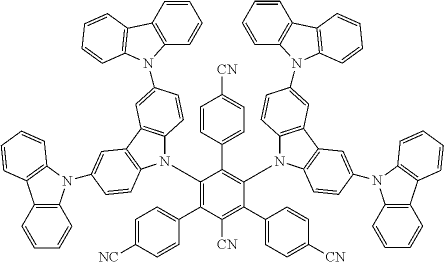 [SMILES: N#Cc1ccc(-c2c(C#N)c(-c3ccc(C#N)cc3)c(-n3c4ccc(-n5c6ccccc6c6ccccc65)cc4c4cc(-n5c6ccccc6c6ccccc65)ccc43)c(-c3ccc(C#N)cc3)c2-n2c3ccc(-n4c5ccccc5c5ccccc54)cc3c3cc(-n4c5ccccc5c5ccccc54)ccc32)cc1]

(4′r,6′R)-4′,6′-bis(9′H-[9,3′: 6′,9″-tercarbazol]-9′-yl)-5′-(4-cyanophenyl)[1,1′:3′,1″-terphenyl]-2′,4,4″-tricarbonitrile (EC-8);

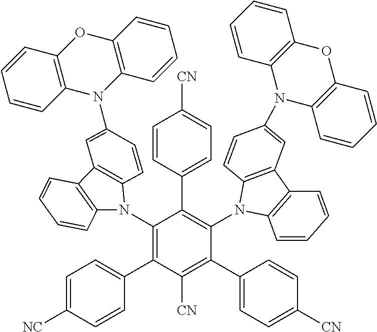 [SMILES: N#Cc1ccc(-c2c(C#N)c(-c3ccc(C#N)cc3)c(-n3c4ccccc4c4cc(N5c6ccccc6Oc6ccccc65)ccc43)c(-c3ccc(C#N)cc3)c2-n2c3ccccc3c3cc(N4c5ccccc5Oc5ccccc54)ccc32)cc1]

(S)-4′,6′-bis(3-(10H-phenoxazin-10-yl)-9H-carbazol-9-yl)-5′-(4-cyanophenyl)[1,1′:3′,1″-terphenyl]-2′,4,4″-tricarbonitrile (EC-9);

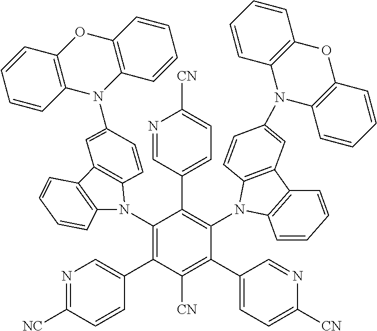 [SMILES: N#Cc1ccc(-c2c(C#N)c(-c3ccc(C#N)nc3)c(-n3c4ccccc4c4cc(N5c6ccccc6Oc6ccccc65)ccc43)c(-c3ccc(C#N)nc3)c2-n2c3ccccc3c3cc(N4c5ccccc5Oc5ccccc54)ccc32)cn1]

(S)-5,5′,5″-(2,4-bis(3-(10H-phenoxazin-10-yl)-9H-carbazol-9-yl)-6-cyanobenzene-1,3,5-triyl)tripicolinonitrile; or (EC-10)

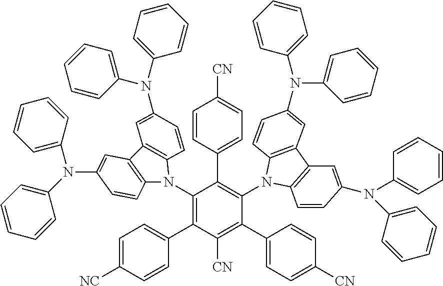 [SMILES: N#Cc1ccc(-c2c(C#N)c(-c3ccc(C#N)cc3)c(-n3c4ccc(N(c5ccccc5)c5ccccc5)cc4c4cc(N(c5ccccc5)c5ccccc5)ccc43)c(-c3ccc(C#N)cc3)c2-n2c3ccc(N(c4ccccc4)c4ccccc4)cc3c3cc(N(c4ccccc4)c4ccccc4)ccc32)cc1]

(s)-4′,6′-bis(3,6-bis(diphenylamino)-9H-carbazol-9-yl)-5′-(4-cyanophenyl)-[1,1′:3′,1″-terphenyl]-2′,4,4″-tricarbonitrile (EC-11).

Some embodiments include a light-emitting element comprising any of the aforementioned compounds, e.g., EC-1, EC-2, EC-3, EC-4, EC-5, EC-6, EC-7, EC-8, EC-9, EC-10, and/or EC-11. In some embodiments, a light-emitting device is provided, comprising any of the aforementioned light-emitting elements. In some embodiments, an emissive layer can comprise any of the aforementioned compounds.

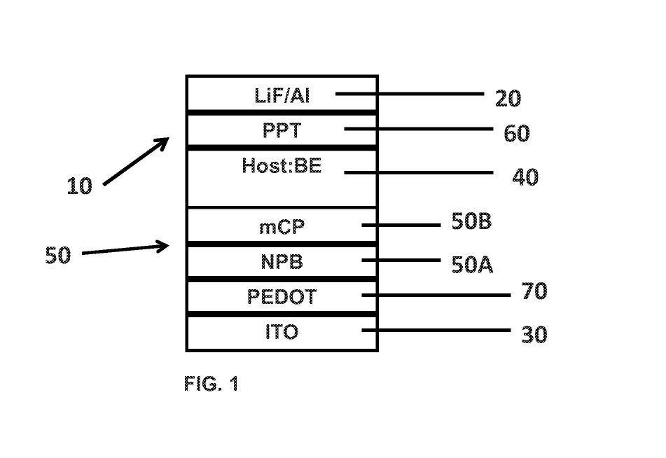

As shown in FIG. 1, there is shown an example embodiment of an organic light-emitting incorporating the compounds of the present application. Organic light-emitting diode device 10 comprises a cathode 20, an anode 30, a light-emitting layer 40 disposed between and electrically connected to the anode and the cathode, a hole-transport layer 50 between the anode and the light-emitting layer 40 and an electron-transport layer 60 between the cathode 20 and the light-emitting layer 40, wherein the light-emitting layer can comprise an emitting compound described herein. In some embodiments, the hole-transport layer 50 can comprise a first hole-transport layer 50A, and a second first hole-transport layer 50B. The second hole transport layer 50B can also function as an electron blocking layer. In some embodiments, second hole transport layer 50B can comprise a high T1 material. In some embodiments, a hole injection layer 70 can be between the anode 30 and the hole transport layer 50. In some embodiments, an electron injection layer can be between the cathode 20 and the electron transport layer 60 (not shown).

An anode layer may comprise a conventional material such as a metal, mixed metal, alloy, metal oxide or mixed-metal oxide, or a conductive polymer. Examples of suitable metals include the Group 1 metals, the metals in Groups 4, 5, 6, and the Group 8-10 transition metals. If the anode layer is to be light-transmitting, mixed-metal oxides of Group 12, 13, and 14 metals or alloys thereof, such as Au, Pt, and indium-tin-oxide (ITO), may be used. The anode layer may include an organic material such as polyaniline, e.g., as described in “Flexible light-emitting diodes made from soluble conducting polymer,” Nature, vol. 357, pp. 477-479 (11 Jun. 1992). Examples of suitable high work function metals include but are not limited to Au, Pt, indium-tin-oxide (ITO), or alloys thereof. In some embodiments, the anode layer can have a thickness in the range of about 1 nm to about 1000 nm.

A cathode layer may include a material having a lower work function than the anode layer. Examples of suitable materials for the cathode layer include those selected from alkali metals of Group 1, Group 2 metals, Group 12 metals including rare earth elements, lanthanides and actinides, materials such as aluminum, indium, calcium, barium, samarium and magnesium, and combinations thereof. Li-containing organometallic compounds, LiF, and Li₂O may also be deposited between the organic layer and the cathode layer to lower the operating voltage. Suitable low work function metals include but are not limited to Al, Ag, Mg, Ca, Cu, Mg/Ag, LiF/Al, CsF, CsF/Al or alloys thereof. In some embodiments, the cathode layer can have a thickness in the range of about 1 nm to about 1000 nm.

In some embodiments, the light-emitting layer may further comprise an emissive component or compound. The emissive component may be a fluorescent and/or a phosphorescent compound. In some embodiments, the emissive component comprises a phosphorescent material. In some embodiments, the emissive component may comprise a dopant. In some embodiments, the dopant is up to about 10% (w/w) of the host, or from about 0.1% (w/w) to about 8% (w/w) of the host, e.g., about 6% (w/w) of the host.

The thickness of the light-emitting layer may vary. In some embodiments, the light-emitting layer has a thickness from about 10 nm to about 200 nm. In some embodiments, the light-emitting layer has a thickness in the range of about 10 nm to about 150 nm, about 20 nm, or about 30 nm.

In some embodiments, the light-emitting layer can further include additional host material. Host materials may be described in co-pending patent applications, e.g., United States Patent Application Publication No. US2012/0193614 (published Aug. 2, 2012, application Ser. No. 13/360,639, filed Jan. 27, 2012); United States Patent Application Publication No. US2012/0179089 (published Jul. 12, 2012, application Ser. No. 13/232,837, filed Sep. 14, 2011); United States Patent Application Publication No. US2011/0251401 (published Oct. 13, 2011, application Ser. No. 13/166,246, filed Jun. 22, 2011); U.S. Pat. No. 8,426,040, issued Apr. 23, 2013, and U.S. Pat. No. 8,263,238, issued Sep. 11, 2012, all of which are incorporated by reference in their entireties for their description of host materials. The host material included in the light-emitting layer can be an optionally substituted compound selected from: an aromatic-substituted amine, an aromatic-substituted phosphine, a thiophene, an oxadiazole, 2-(4-biphenylyl)-5-(4-tert-butylphenyl)-1,3,4-oxadiazole (PBD), 1,3-bis(N,N-t-butyl-phenyl)-1,3,4-oxadiazole (OXD-7), a triazole, 3-phenyl-4-(1′-naphthyl)-5-phenyl-1,2,4-triazole (TAZ), 3,4,5-Triphenyl-1,2,3-triazole, 3,5-Bis(4-tert-butyl-phenyl)-4-phenyl[1,2,4]triazole, an aromatic phenanthroline, 2,9-dimethyl-4,7-diphenyl-phenanthroline (bathocuproine or BCP), 2,9-Dimethyl-4,7-diphenyl-1,10-phenanthroline, a benzoxazole, a benzothiazole, a quinoline, aluminum tris(8-hydroxyquinolate) (Alq₃), a pyridine, a dicyanoimidazole, cyano-substituted aromatic, 1,3,5-tris(2-N-phenylbenzimidazolyl)benzene (TPBI), 4,4′-bis[N-(naphthyl)-N-phenyl-amino]biphenyl (α-NPD), N,N′-bis(3-methylphenyl)N,N′-diphenyl-[1,1′-biphenyl]-4,4′-diamine (TPD), 4,4′-bis[N,N′-(3-tolyl)amino]-3,3′-dimethylbiphenyl (M14), 4,4′-bis[N,N′-(3-tolyl)amino]-3,3′-dimethylbiphenyl (HMTPD), 1,1-Bis(4-bis(4-methylphenyl) aminophenyl) cyclohexane, a carbazole, 4,4′-N,N′-dicarbazole-biphenyl (CBP), poly(9-vinylcarbazole) (PVK), N,N′N″-1,3,5-tricarbazoloylbenzene (tCP), a polythiophene, a benzidine, N,N′-bis(4-butylphenyl)-N,N′-bis(phenyl)benzidine, a triphenylamine, 4,4′,4″-Tris(N-(naphthylen-2-yl)-N-phenylamino)triphenylamine, 4,4′,4″-tris(3-methylphenylphenylamino)triphenylamine (MTDATA), dibenzo[b,d]thiophene-2,8-diylbis(diphenylphosphine oxide) (PPT), 3,3′-di(9H-carbazol-9-yl)-1,1′-biphenyl (mCBP), a phenylenediamine, a polyacetylene, and a phthalocyanine metal complex.

In some embodiments, the light-emitting device may further comprise a hole-transport layer between the anode and the light-emitting layer and an electron-transport layer between the cathode and the light-emitting layer. In some embodiments, all of the light-emitting layer, the hole-transport layer and the electron-transport layer comprise the host compound described herein.

In some embodiments, the hole-transport layer may comprise a hole-transfer material. Suitable hole-transport materials are known to those skilled in the art. Exemplary hole-transport materials include: 1,1-Bis(4-bis(4-methylphenyl) aminophenyl) cyclohexane; 2,9-Dimethyl-4,7-diphenyl-1,10-phenanthroline; 3,5-Bis(4-tert-butyl-phenyl)-4-phenyl[1,2,4]triazole; 3,4,5-Triphenyl-1,2,3-triazole; 4,4′,4″-Tris(N-(naphthylen-2-yl)-N-phenylamino)triphenylamine; 4,4′,4′-tris(3-methylphenylphenylamino)triphenylamine (MTDATA); 4,4′-bis[N-(naphthyl)-N-phenyl-amino]biphenyl (α-NPD); 4,4′-bis[N,N′-(3-tolyl)amino]-3,3′-dimethylbiphenyl (HMTPD); 4,4′-bis[N,N′-(3-tolyl)amino]-3,3′-dimethylbiphenyl (M14); 4,4′-N,N′-dicarbazole-biphenyl (CBP); 1,3-N,N-dicarbazole-benzene (mCP); poly(9-vinylcarbazole) (PVK); a benzidine; a carbazole; a phenylenediamine; a phthalocyanine metal complex; a polyacetylene; a polythiophene; a triphenylamine; an oxadiazole; copper phthalocyanine; N,N′-bis(3-methylphenyl)N,N′-diphenyl-[1,1′-biphenyl]-4,4′-diamine (TPD); N,N′N″-1,3,5-tricarbazoloylbenzene (tCP); N,N′-bis(4-butylphenyl)-N,N′-bis(phenyl)benzidine; 4,4′-bis[N-(1-napthyl)-N-phenylamino]biphenyl (NPB), 4,4′4″-tri(N-carbazolyl)triphenylamine (TcTa); and the like.

In some embodiments, the electron-transport layer may comprise a electron-transfer material. Suitable electron transport materials are known to those skilled in the art.

Exemplary electron transport materials that can be included in the electron transport layer are an optionally substituted compound selected from: aluminum tris(8-hydroxyquinolate) (Alq₃), 2-(4-biphenylyl)-5-(4-tert-butylphenyl)-1,3,4-oxadiazole (PBD), 1,3-bis(N,N-t-butyl-phenyl)-1,3,4-oxadiazole (OXD-7), 1,3-bis[2-(2,2′-bipyridine-6-yl)-1,3,4-oxadiazo-5-yl]benzene (BPY-OXD), 3-phenyl-4-(1′-naphthyl)-5-phenyl-1,2,4-triazole (TAZ), 2,9-dimethyl-4,7-diphenyl-phenanthroline (bathocuproine or BCP), and 1,3,5-tris[2-N-phenylbenzimidazol-z-yl]benzene (TPBI). In one embodiment, the electron transport layer is aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI), or a derivative or a combination thereof.

If desired, additional layers may be included in the light-emitting device. Additional layers that may be included include an electron injection layer (EIL), hole blocking layer (HBL), exciton blocking layer (EBL), and/or hole injection layer (HIL). In addition to separate layers, some of these materials may be combined into a single layer. In addition to separate layers, some of these materials may be combined into a other layers, e.g., an electron blocking layer and/or an exciton blocking layer can be combined into a hole transport layer; or a hole blocking layer and/or an exciton blocking layer can be combined into an electron transport layer.

In some embodiments, the light-emitting device can include an electron injection layer between the cathode layer and the light-emitting layer. In some embodiments, the lowest un-occupied molecular orbital (LUMO) energy level of the material(s) that can be included in the electron injection layer is high enough to prevent it from receiving an electron from the light-emitting layer. In other embodiments, the energy difference between the LUMO of the material(s) that can be included in the electron injection layer and the work function of the cathode layer is small enough to allow efficient electron injection from the cathode. A number of suitable electron injection materials are known to those skilled in the art. Examples of suitable material(s) that can be included in the electron injection layer include but are not limited to, an optionally substituted compound selected from the following: aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI) a triazine, a metal chelate of 8-hydroxyquinoline such as tris(8-hydroxyquinolate) aluminum, and a metal thioxinoid compound such as bis(8-quinolinethiolato) zinc. In one embodiment, the electron injection layer is aluminum quinolate (Alq₃), 2-(4-biphenylyl)-5-(4-tert-butylphenyl)-1,3,4-oxadiazole (PBD), phenanthroline, quinoxaline, 1,3,5-tris[N-phenylbenzimidazol-z-yl]benzene (TPBI), or a derivative thereof, or a combination thereof.

In some embodiments, the device can include a hole blocking layer, e.g., between the cathode and the light-emitting layer. Various suitable hole blocking materials that can be included in the hole blocking layer are known to those skilled in the art. Suitable hole blocking material(s) include but are not limited to, an optionally substituted compound selected from the following: bathocuproine (BCP), 3,4,5-triphenyl-1,2,4-triazole, 3,5-bis(4-tert-butyl-phenyl)-4-phenyl-[1,2,4]triazole, 2,9-dimethyl-4,7-diphenyl-1,10-phenanthroline, and 1,1-bis(4-bis(4-methylphenyl)aminophenyl)-cyclohexane.

In some embodiments, the light-emitting device can include an exciton blocking layer, e.g., between the light-emitting layer and the anode. In one embodiment, the band gap of the material(s) that comprise exciton blocking layer is large enough to substantially prevent the diffusion of excitons. A number of suitable exciton blocking materials that can be included in the exciton blocking layer are known to those skilled in the art. Examples of material(s) that can compose an exciton blocking layer include an optionally substituted compound selected from the following: aluminum quinolate (Alq₃), 4,4′-bis[N-(naphthyl)-N-phenyl-amino]biphenyl (α-NPD), 4,4′-N,N′-dicarbazole-biphenyl (CBP), and bathocuproine (BCP), and any other material(s) that have a large enough band gap to substantially prevent the diffusion of excitons.

In some embodiments, the light-emitting device can include a hole injection layer, e.g., between the light-emitting layer and the anode. Various suitable hole injection materials that can be included in the hole injection layer are known to those skilled in the art. Exemplary hole injection material(s) include an optionally substituted compound selected from the following: a polythiophene derivative such as poly(3,4-ethylenedioxythiophene (PEDOT)/polystyrene sulphonic acid (PSS), a benzidine derivative such as N,N,N′,N′-tetraphenylbenzidine, poly(N,N′-bis(4-butylphenyl)-N,N′-bis(phenyl)benzidine), a triphenylamine or phenylenediamine derivative such as N,N′-bis(4-methylphenyl)-N,N′-bis(phenyl)-1,4-phenylenediamine, 4,4′,4″-tris(N-(naphthylen-2-yl)-N-phenylamino)triphenylamine, an oxadiazole derivative such as 1,3-bis(5-(4-diphenylamino)phenyl-1,3,4-oxadiazol-2-yl)benzene, a polyacetylene derivative such as poly(1,2-bis-benzylthio-acetylene), and a phthalocyanine metal complex derivative such as phthalocyanine copper. Hole-injection materials, while still being able to transport holes, may have a hole mobility substantially less than the hole mobility of conventional hole transport materials.

Those skilled in the art would recognize that the various materials described above can be incorporated in several different layers depending on the configuration of the device. In one embodiment, the materials used in each layer are selected to result in the recombination of the holes and electrons in the light-emitting layer.

Light-emitting devices comprising the compounds disclosed herein can be fabricated using techniques known in the art, as informed by the guidance provided herein. For example, a glass substrate can be coated with a high work functioning metal such as ITO which can act as an anode. After patterning the anode layer, a light-emitting layer that includes at least a compound disclosed herein can be deposited on the anode. The cathode layer, comprising a low work functioning metal (e.g., Mg:Ag), can then be vapor evaporated onto the light-emitting layer. If desired, the device can also include an electron transport/injection layer, a hole blocking layer, a hole injection layer, an exciton blocking layer and/or a second light-emitting layer that can be added to the device using techniques known in the art, as informed by the guidance provided herein.

The following embodiments are contemplated:

### Embodiment 1

An emissive compound represented by Formula 1:

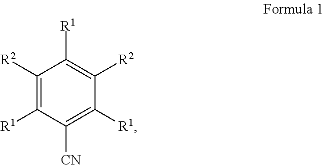

wherein each R¹ is independently an optionally substituted phenyl, optionally substituted pyridinyl, or optionally substituted pyrimidinyl; and

each R² is independently an optionally substituted carbazolyl, an optionally substituted benzocarbazolyl, an optionally substituted phenoxazinyl, an optionally substituted phenothiazinyl, or an optionally substituted dihydrophenazinyl.

### Embodiment 2

The compound of embodiment 1, wherein any substituent present has a molecular weight of 15 Daltons to 500 Daltons.

### Embodiment 3

The compound of embodiment 2, wherein R¹ is optionally substituted phenyl.

### Embodiment 4

The compound of embodiment 2, wherein R¹ is optionally substituted pyridin-3-yl.

### Embodiment 5

The compound of embodiment 2, wherein R² is optionally substituted carbazolyl.

### Embodiment 6

The compound of embodiment 2, wherein R² is optionally substituted benzocarbazolyl.

### Embodiment 7

The compound of embodiment 1, wherein R¹ is selected from

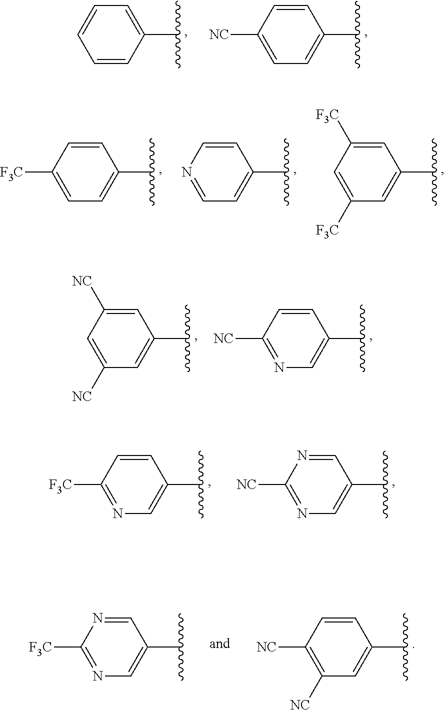

### Embodiment 8

The compound of embodiment 1, wherein R² is selected from

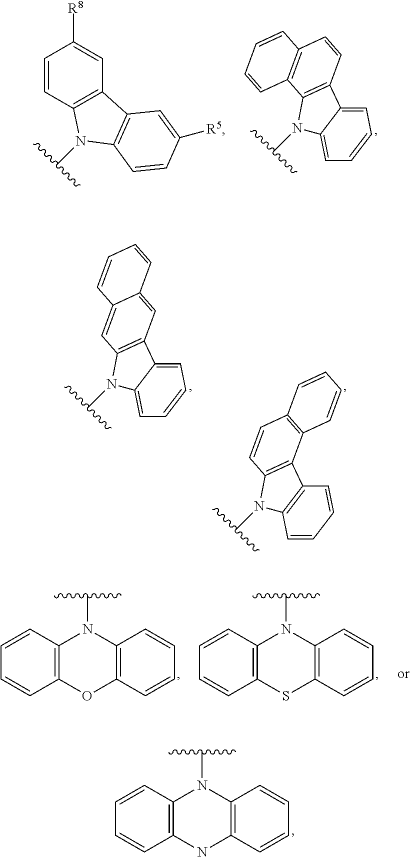

wherein each R⁸ and R⁵ are independently optionally substituted phenyl, optionally substituted amine, optionally substituted carbazolyl, optionally substituted phenoxazinyl, optionally substituted phenothiazinyl, optionally substituted dihydrophenazinyl, a C₁-C₃ alkyl or hydrogen.

### Embodiment 9

The compound of embodiment 1, wherein the compound is selected from:

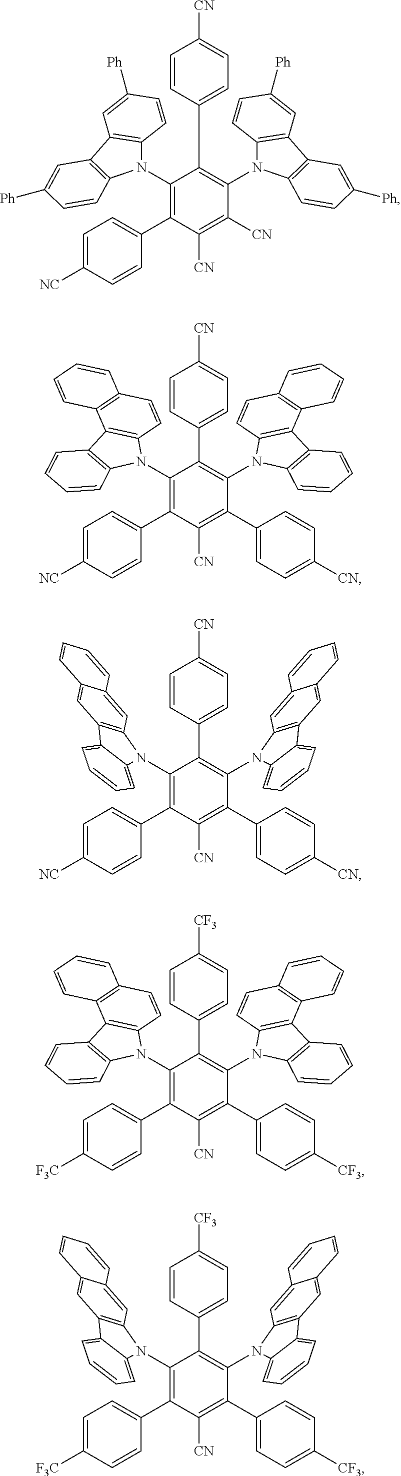

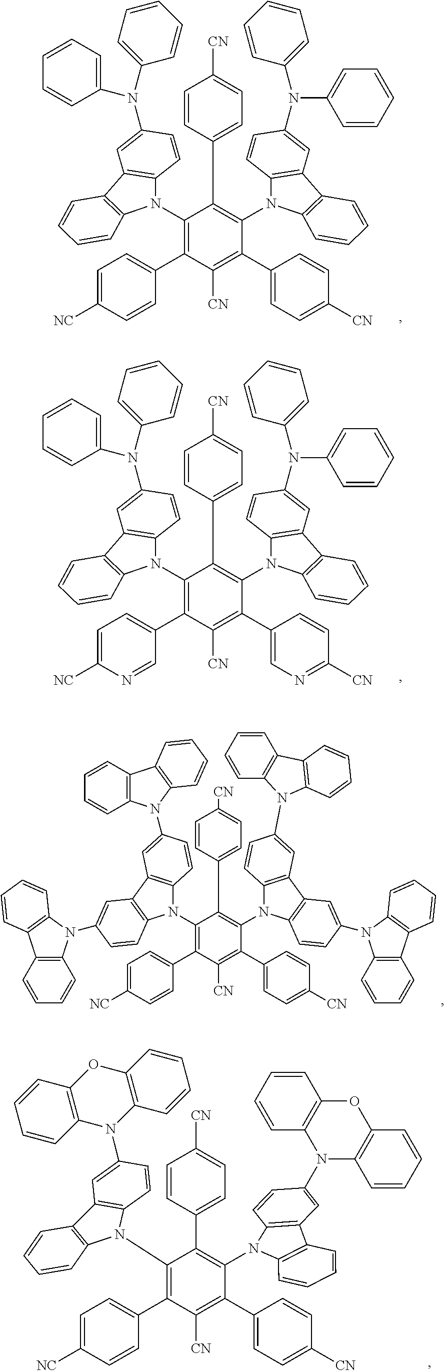

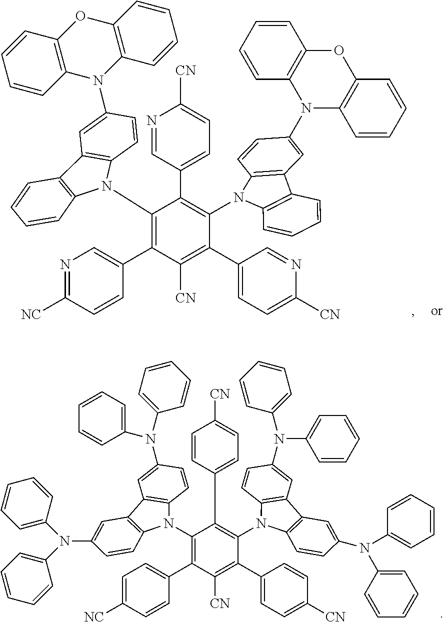 [SMILES: N#Cc1ccc(-c2c(C#N)c(-c3ccc(C#N)nc3)c(-n3c4ccccc4c4cc(N5c6ccccc6Oc6ccccc65)ccc43)c(-c3ccc(C#N)nc3)c2-n2c3ccccc3c3cc(N4c5ccccc5Oc5ccccc54)ccc32)cn1 ; N#Cc1ccc(-c2c(C#N)c(-c3ccc(C#N)cc3)c(-n3c4ccc(N(c5ccccc5)c5ccccc5)cc4c4cc(N(c5ccccc5)c5ccccc5)ccc43)c(-c3ccc(C#N)cc3)c2-n2c3ccc(N(c4ccccc4)c4ccccc4)cc3c3cc(N(c4ccccc4)c4ccccc4)ccc32)cc1]

### Embodiment 10

The compound of embodiment 1, wherein the compound is fluorescent or phosphorescent.

### Embodiment 11

The compound of embodiment 1, wherein the compound is electroluminescent.

### Embodiment 12

A light-emitting element comprising the compound of embodiment 1.

### Embodiment 13

A light-emitting device comprising the light-emitting element of embodiment 12.

### Embodiment 14

An organic light-emitting diode device comprising:
a cathode;
an anode; and
a light-emitting layer disposed between and electrically connected to the anode and the cathode, wherein the light-emitting layer comprises an emitting compound according to embodiment 2.

### Embodiment 15

The device of embodiment 14, further comprising a hole-transport layer between the anode and the light-emitting layer and an electron-transport layer between the cathode and the light-emitting layer.

### Embodiment 16

The device of embodiment 14, wherein R¹ is optionally substituted phenyl.

### Embodiment 17

The device of embodiment 14, wherein R¹ is optionally substituted pyridin-3-yl.

### Embodiment 18

The device of embodiment 14, wherein R² is optionally substituted carbazolyl.

### Embodiment 19

The device of embodiment 14, wherein R² is optionally substituted benzocarbazolyl.

### Embodiment 20

A compound represented by the formula

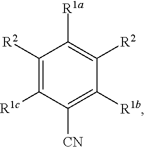

wherein R^{1a} is

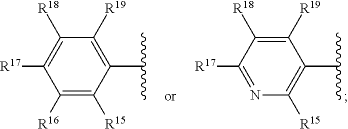

and

wherein R^{1b} is

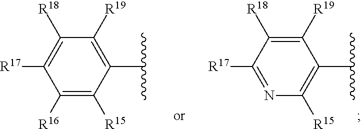

and.

wherein R^{1c} is

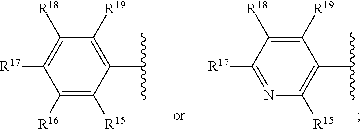

and

each R² is independently

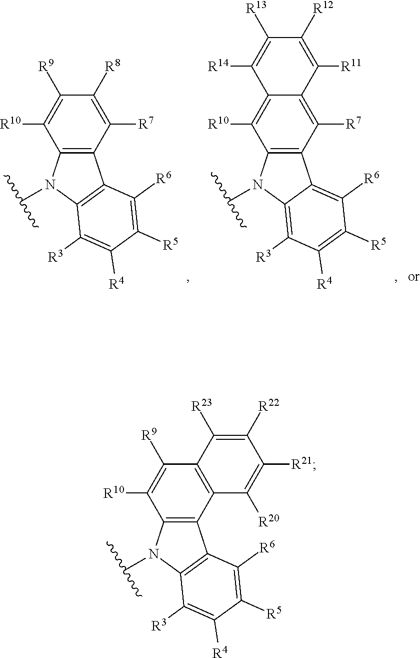

wherein R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R²³, are independently H or any substituent, such as a substituent having from 0 to 6 carbon atoms and from 0 to 5 heteroatoms, wherein each heteroatom is independently: O, N, P, S, F, Cl, Br, or I; and/or having a molecular weight of 15 g/mol to 300 g/mol.

### Embodiment 21

The compound of embodiment 20, wherein R⁸ is optionally substituted carbazolyl.

### Embodiment 22

The compound of embodiment 20, wherein R⁵ is optionally substituted carbazolyl.

### Embodiment 23

The compound of embodiment 20, wherein R⁸ is optionally substituted phenyl.

### Embodiment 24

The compound of embodiment 20, wherein R⁸ is optionally substituted diphenylamino.

### Embodiment 25

The compound of embodiment 20, wherein R⁸ is phenoxazinyl.

### Embodiment 26

The compound of embodiment 20, wherein R¹⁷ is CN.

### Embodiment 27

The compound of embodiment 20, wherein R¹⁷ is CF₃.

### EXAMPLES

### Example 1

### Luminescent Dye

### Synthesis of Bipolar Hosts

### 8-1. Example of Synthesis

### Example 1.1

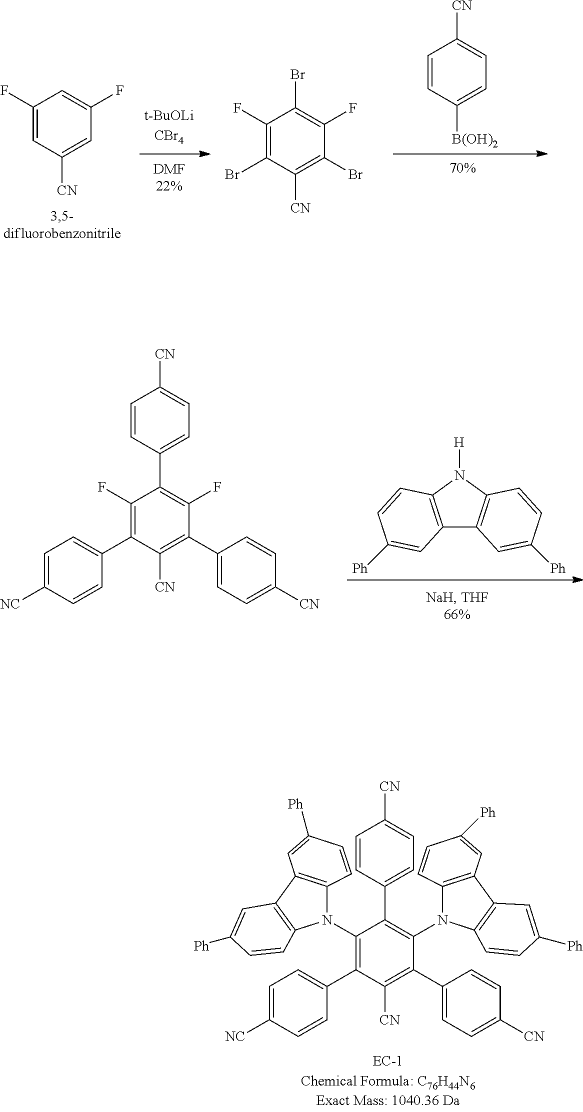

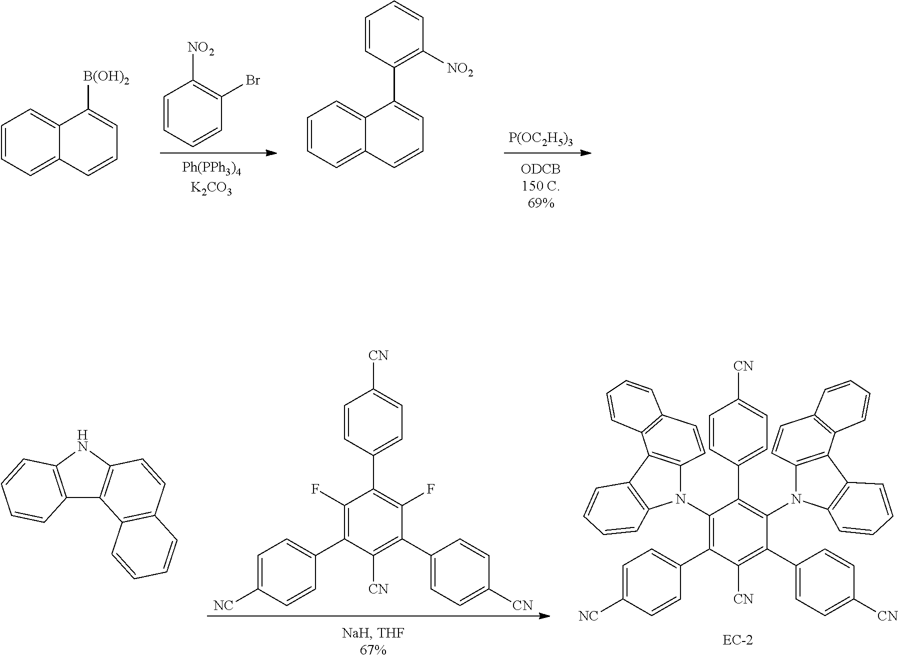

8.1.1

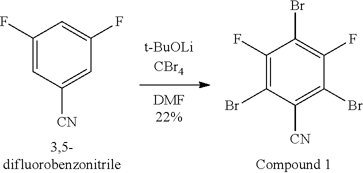 [SMILES: N#Cc1cc(F)cc(F)c1 ; N#Cc1c(Br)c(F)c(Br)c(F)c1Br]

### 2,4,6-tribromo-3,5-difluorobenzonitrile (Compound 1)

To a solution of 3,5-difluorobenzonitrile (6.95 g, 0.050 mol), carbon tetrabromide (CBr₄) (58 g, 0.17 mol) in DMF/xylene (50 mL/50 mL) was added lithium tert-butoxide (t-BuOLi) powder slowly under argon. The resulting mixture was heated at 60° C. for about 4 hours, then diluted with toluene (300 mL), washed with brine, dried over Na₂SO₄, concentrated to 30 mL, then dissolved in dichloromethane (DCM) and purified by flash column using eluents of hexanes/dichloromethane (4:1). The desired fractions were collected, and the impure fractions were purified again by flash column using eluents of dichloromethane/hexanes (10% to 30%). After removal of solvents, a white solid (Compound 1) was obtained (3.85 g, in about 22% yield).

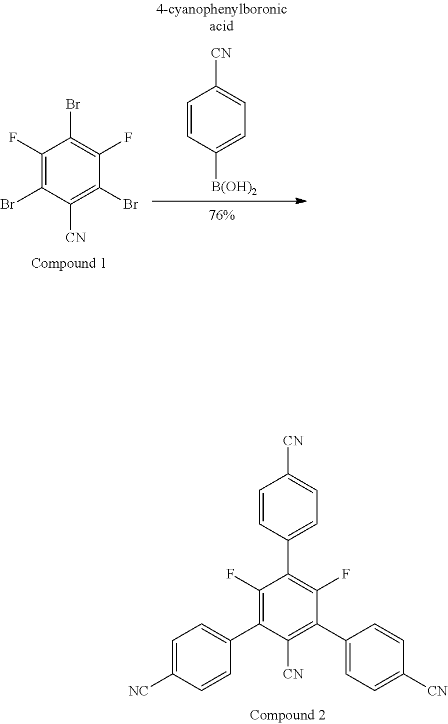 [SMILES: N#Cc1c(Br)c(F)c(Br)c(F)c1Br ; N#Cc1ccc(B(O)O)cc1 ; N#Cc1ccc(-c2c(F)c(-c3ccc(C#N)cc3)c(C#N)c(-c3ccc(C#N)cc3)c2F)cc1]

### 5′-(4-cyanophenyl)-4′,6′-difluoro-[1,1′:3′,1″-terphenyl]-2′,4,4″-tricarbonitrile (Compound 2)

A mixture of 4,6-tribromo-3,5-difluorobenzonitrile (Compound 1) (1.88 g, 5 mmol), 4-cyanophenylboronic acid (5.94 g, 26 mmol), Tris(dibenzylideneacetone) dipalladium(0) (Pd₂(dba)₃) (0.55 g, 0.6 mmol), Dicyclohexylphosphino-2′,6′-dimethoxy biphenyl (SPhos) (0.492 g, 1.2 mmol) and potassium phosphate (K₃PO₄) (5.75 g, 25 mmol) in anhydrous toluene was degassed, heated at 120° C. for about 16 hours. The resulting mixture was worked up with ethyl acetate/brine. The organic phase was collected and dried over Na₂SO₄, loaded on silica gel, purified by flash column using eluents of hexanes/ethyl acetate (90% to 70%). The desired fractions were collected, concentrated and recrystallized in dichloromethane/hexanes to give a solid (Compound 2) (1.68 g, in 76% yield).

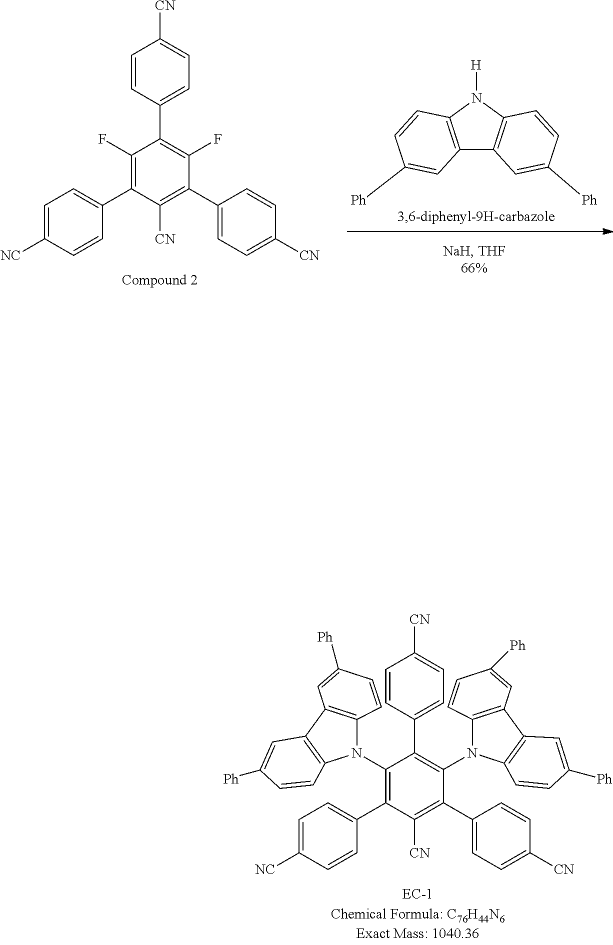

### Compound EC-1

To a mixture of 5′-(4-cyanophenyl)-4′,6′-difluoro-[1,1′:3′,1″-terphenyl]-2′,4,4″-tricarbonitrile (Compound 2) (0.30 g, 0.68 mmol) and 3,6-diphenyl-9H-carbazole (0.67 g, 2.0 mmol) in anhydrous tetrahydrofuran (THF) (15 mL), was added sodium hydride (NaH) (60% in mineral oil, 0.16 g, 4 mmol) at about 0° C. The mixture was stirred at room temperature for about 1.5 hour, then heated at about 60° C. for about 16 hours. The whole was worked up with dichloromethane/brine, dried over Na₂SO₄, loaded on silica gel and purified by flash column using eluents of dichloromethane/hexanes (20% to 100%). The desired fractions were collected, concentrated, recrystallized in dichloromethane/hexanes to give a light yellow solid (0.467 g, in 66% yield). Confirmed by LCMS (APCI): calculated for C₇₀H₅N₆(M+H): 1041. found: 1041.

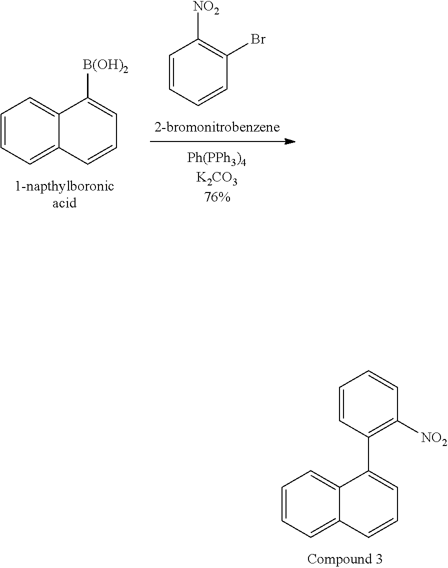 [SMILES: OB(O)c1cccc2ccccc12 ; O=[N+]([O-])c1ccccc1Br ; O=[N+]([O-])c1ccccc1-c1cccc2ccccc12]

### 1-(2-nitrophenyl)naphthalene (Compound 3)

A mixture of 2-bromonitrobenzene (10.00 g, 49.5 mmol, 1.00 eq), 1-napthylboronic acid (16.18 g, 94.0 mmol, 1.90 eq), tetrakis(triphenylphosphine)palladium (Pd(PPh₃)₄) (4.0 g, 3.47 mmol, 0.07 eq), and 1,4-dioxane (400.0 mL) were degassed with bubbling argon for about 1.5 hours at room temperature. Aqueous potassium carbonate (K₂CO₃) (27.37 g, 198.0 mmol, 4.0 eq K₂CO₃ in 80.0 mL of water) was then added and the reaction was degassed for about an additional 30 minutes. The reaction mixture was then heated to about 90° C. overnight, maintaining an argon atmosphere. Once complete and cooled to room temperature, an aqueous workup was performed using ethyl acetate, water, and brine, and the organic phase was dried by magnesium sulfate. The crude material was then purified by flash chromatography using the following eluent gradient (% dichloromethane in hexanes over column volumes (CV)): linear from 10% to 15% over 7 CV, linear from 15% to 20% over 2 CV, isocratic at 20% until the product was fully eluted. The product fractions were concentrated to yield desired compound (12.0 g, in 76% yield) as a light yellow solid (Compound 3). Confirmed by LCMS and ¹H NMR.

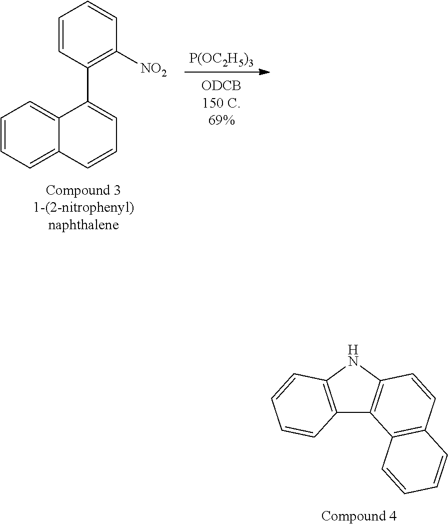

### 7H-benzo[c]carbazole (Compound 4)

To a solution of 1-(2-nitrophenyl)naphthalene (Compound 3) (4.5 g, 18 mmol) in 1,2-dichlorobenzene (20 mL) was added triethylphosphite (P(OC₂H₅)₃) (20 mL, 144 mmol). The solution was heated at about 150° C. for about 15 hours. After removal of solvent and excess reagent by vacuum distillation, the remaining was dissolved in dichloromethane/hexanes (20 mL/20 mL) and purified by flash column using eluents of hexanes to hexanes/dichloromethane (4:1). The desired fractions were collected, concentrated, loaded on silica gel and purified by flash column again using eluents of dichloromethane/hexanes (10% to 20%). Removal of solvents gives a white solid (Compound 4) (2.70 g, in 69% yield).

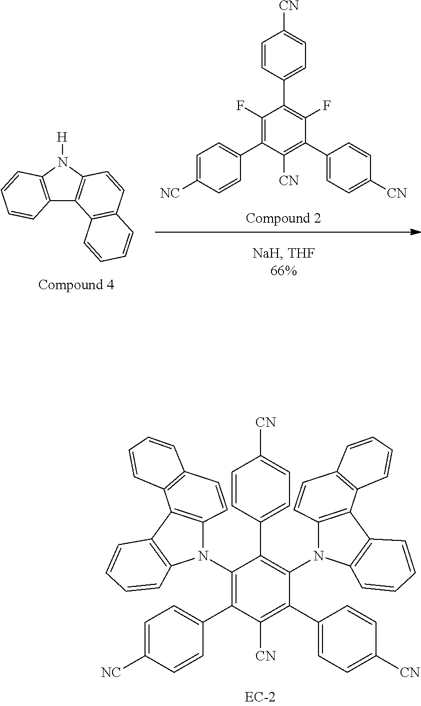

### Compound EC-2

To a solution of 7H-benzo[c]carbazole (Compound 4) (0.217 g, 1.0 mmol), 5′-(4-cyanophenyl)-4′,6′-difluoro-[1,1′:3′,1″-terphenyl]-2′,4,4″-tricarbonitrile (Compound 2) (0.166 g, 0.375 mmol) in anhydrous THF (10 mL) was added sodium hydride (60% in mineral oil, 0.08 g, 2 mmol) at about 0° C. The whole was stirred at room temperature for about two hours, then heated at about 55° C. for about 15 hours. The resulting mixture was worked up with dichloromethane/brine. After dried over Na₂SO₄, the organic was loaded on silica gel and purified by flash column using eluents of dichloromethane/hexanes (20% to 100%). The desired fractions were collected, concentrated, recrystallized in dichloromethane/hexanes to give a yellow solid (EC-2) (0.21 g, in 67% yield). Confirmed by LCMS (APCI): calculated for C₆₀H₃₃N₆ (M+H): 837. Found: 837.

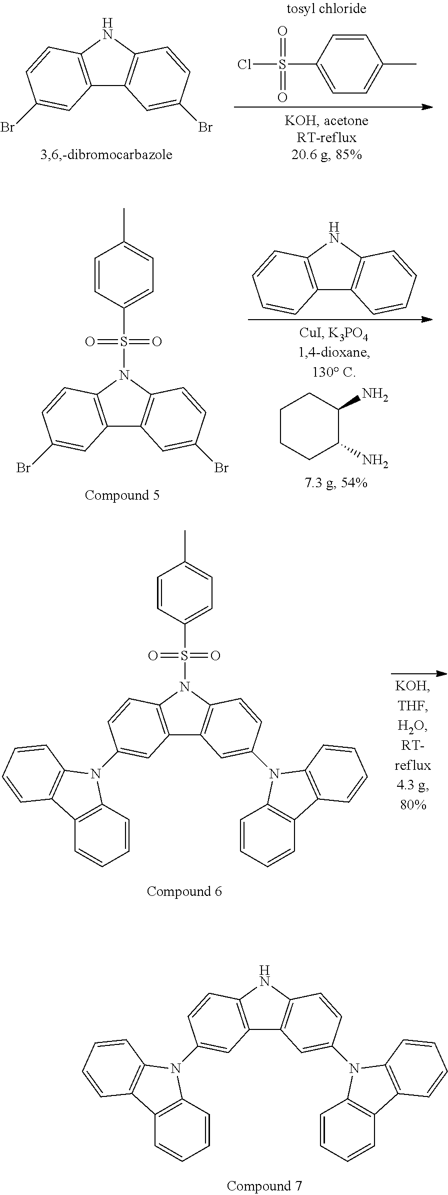

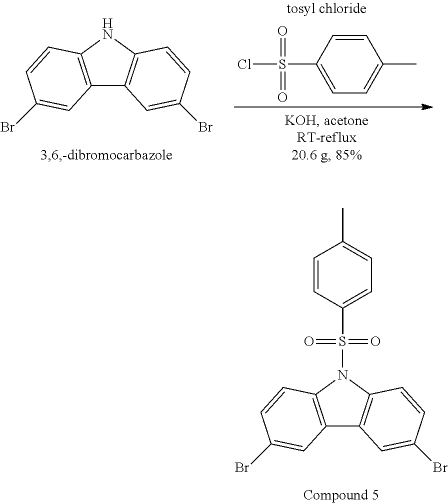 [SMILES: Brc1ccc2[nH]c3ccc(Br)cc3c2c1 ; Cc1ccc(S(=O)(=O)Cl)cc1 ; Cc1ccc(S(=O)(=O)n2c3ccc(Br)cc3c3cc(Br)ccc32)cc1]

### 3,6-dibromo-N-tosylcarbazole (Compound 5)

To a solution of 3,6-dibromocarbazole (16.25 g, 50.00 mmol), and potassium hydroxide (3.93 g, 70.00 mmol) in anhydrous acetone (100.00 mL), tosyl chloride (13.35 g, 70.00 mmol) was added in 4 g portions at room temperature. The reaction was then heated at reflux for about 3 hours. The resulting mixture was then poured into stirring water (300.00 mL) while still hot. The precipitate was filtered, washed with methanol, and dried. It was then recrystallized from dichloromethane/methanol to give compound 5 as a white solid, 20.6 g, 82% yield. Confirmed by ¹HNMR.

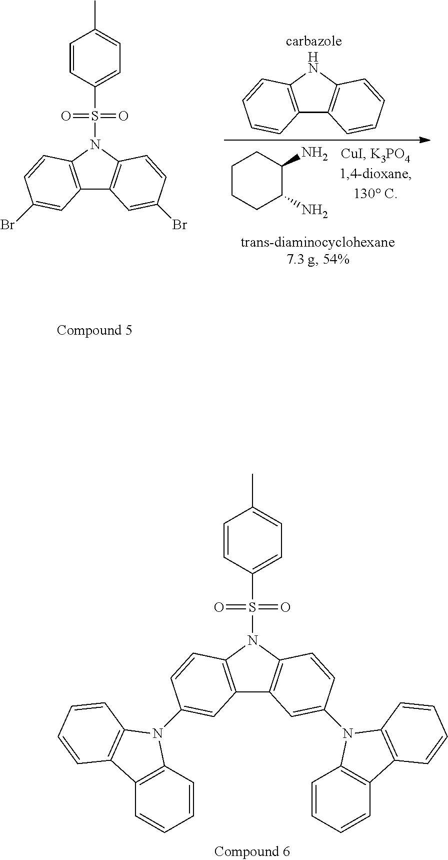

### 3,6-bis(N-carbazolyl)-N-tosylcarbazole (compound 6)

A mixture of compound 5 (10.00 g, 20.87 mmol), carbazole (13.37 g, 79.93 mmol), copper iodide (1.59 g, 8.33 mmol), trans-diaminocyclohexane (1.0 mL, 8.34 mmol), and potassium phosphate (19.21 g, 90.51 mmol) in anhydrous dioxane (165.00 mL) was degassed with bubbling argon for 2 hours at room temperature. The mixture was then heated at 120° C. for 16 hours. An aqueous workup was performed with ethyl acetate and brine, dried with magnesium sulfate. The crude product was passed through a silica gel plug to remove the copper catalyst, using dichloromethane as the eluent. The semi-crude product was then purified by silica gel column using dichloromethane/hexanes eluent in a linear gradient of 25% to 70%, holding at 70% until the product fully eluted. The product fractions were then recrystallized twice from dichloromethane in methanol to yield compound 6 as a white solid, 7.3 g, 54% yield. Confirmed by LCMS (APCI): calculated for C₄₃H₃₀N₃O₂S (M+H): 652. Found: 652.

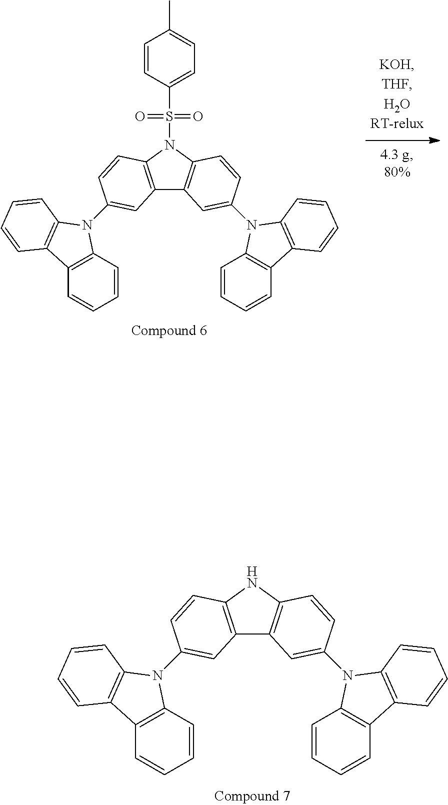 [SMILES: Cc1ccc(S(=O)(=O)n2c3ccc(-n4c5ccccc5c5ccccc54)cc3c3cc(-n4c5ccccc5c5ccccc54)ccc32)cc1 ; c1ccc2c(c1)c1ccccc1n2-c1ccc2[nH]c3ccc(-n4c5ccccc5c5ccccc54)cc3c2c1]

### 3,6-bis(N-carbazolyl)carbazole (compound 7)

To a solution of compound 6 in tetrahydrofuran/methanol (90.00 mL/35.00 mL), sodium hydroxide (6.08 g, 151.93 mmol) dissolved in water (35.00 mL) was added, and the resulting mixture was heated at 80° C. for 18 hours. An aqueous workup was performed with ethyl acetate and brine, and was dried with magnesium sulfate. The material was purified by recrystallization by dissolving in dichloromethane and adding an equal volume of methanol before concentrating. The resulting solid was collected to yield compound 7 as a white solid, 4.3 g, 80% yield. Confirmed by LCMS (APCI): calculated for C₃H₂₄N₃ (M+H): 498. Found: 498.

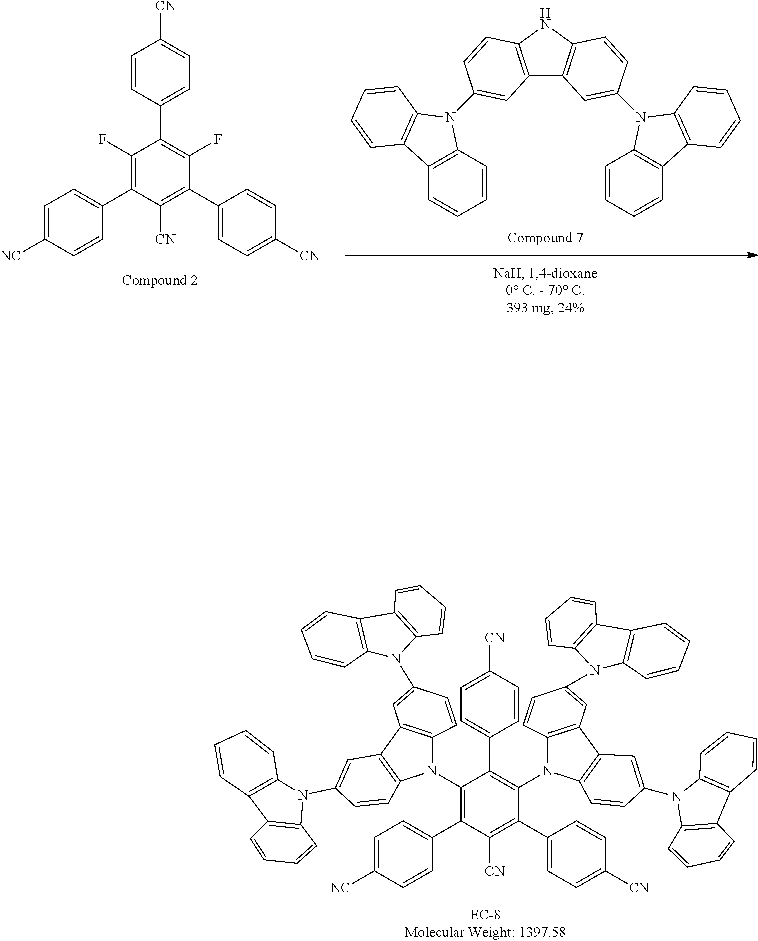

### Compound EC-8

To a solution of 2 (1.46 g, 2.94 mmol), 5′-(4-cyanophenyl)-4′,6′-difluoro-[1,1′:3′,1″-terphenyl]-2′,4,4″-tricarbonitrile (Compound 7) (0.52 g, 1.18 mmol) in anhydrous THF (45.00 mL) was added sodium hydride (60% in mineral oil, 0.099 g, 4.11 mmol) at about 0° C. The resulting mixture was stirred at about 0° C. for about 1.5 hours, then was heated at about 75° C. for about 15 hours. An aqueous workup was performed with ethyl acetate and brine, and the organic phase was dried with sodium sulfate. The crude material was purified 5 times by silica gel column, using the following eluents: toluene (column 1), 25% to 35% ethyl acetate in hexanes (column 2), 1.5% ethyl acetate in toluene (column 3), 1% to 1.25% ethyl acetate in toluene (column 4), 1% to 2% ethyl acetate in toluene (column 5). The product was then recrystallized twice from dichloromethane/methanol to yield EC-8 as a yellow solid, 0.39 g, 24% yield. Confirmed by ¹HNMR.

### 8.2. Physical Properties and OLED Device Data of the Materials

EC-1 (2 mg) was dissolved in 1 mL of 2-methyltetrahydrofuran (2-MeTHF) and then the resulting solution was transferred into quartz tube. Then the quartz tube containing EC-1 was frozen (77 K) by liquid nitrogen prior to measurement. Triplet (T₁) energy was measured by phosphorescent emission spectrum at 77 K, using Fluoromax-3 spectrophotometer (Horiba Instruments, Irvine Calif., USA). EC-2 samples were tested in a similar manner, the results of which are provided below in Table 1.

TABLE 1
Physical data of the emissive materials

| | | | S₁—T₁ | | PE | LE | EQE |
|---|---|---|---|---|---|---|---|
| | Structure | ₑₘ (nm) | (eV) | _{PL} (N₂) | (lm/w) | (cd/A) | (@1k nit) |
| EC-1 | 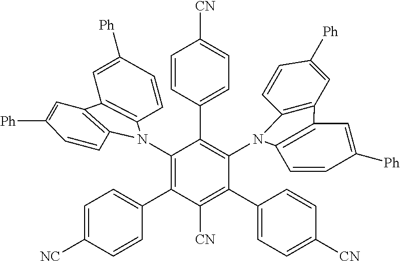 [SMILES: N#Cc1ccc(-c2c(C#N)c(-c3ccc(C#N)cc3)c(-n3c4ccc(-c5ccccc5)cc4c4cc(-c5ccccc5)ccc43)c(-c3ccc(C#N)cc3)c2-n2c3ccc(-c4ccccc4)cc3c3cc(-c4ccccc4)ccc32)cc1] | 491 | <0.01 | 99.7% | 23 | 47 | 18.5% |
| EC-2 | 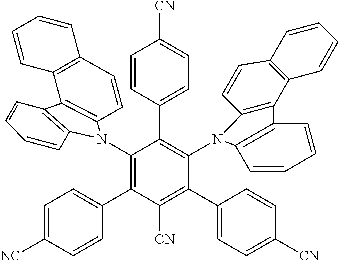 [SMILES: N#Cc1ccc(-c2c(C#N)c(-c3ccc(C#N)cc3)c(-n3c4ccccc4c4c5ccccc5ccc43)c(-c3ccc(C#N)cc3)c2-n2c3ccccc3c3c4ccccc4ccc32)cc1] | 496 | <0.01 | 100% | — | — | — |

### 8-3. Example of OLED Device Configuration and Performance

### Example 2.1 (Device-A)

### Fabrication of Light-Emitting Device

A device was fabricated in the following manner. The ITO substrates having sheet resistance of about 14 ohm/sq were cleaned ultrasonically and sequentially in detergent, water, acetone and then iso-propyl alcohol; and then dried in an oven at about 80° C. for about 30 min under ambient environment. Substrates were baked at about 200° C. for about 1 hour in an ambient environment, then under UV-ozone treatment for about 30 minutes. PEDOT:PSS (hole-injection material) was then spin-coated on the annealed substrate at about 4000 rpm for about 30 sec. The coated layer was then baked at about 100° C. for 30 min in an ambient environment, followed by baking at 200° C. for 30 min inside a glove box (N₂ environment). The substrate was then be transferred into a vacuum chamber, where N,N′-bis(naphthalene-1-yl)-N,N′-bis(phenyl)-benzidine (NPB) [hole transporting material]) was vacuum deposited at a rate of about 0.1 nm/s rate under a base pressure of about 2×10⁻⁷ Torr. 1,3-bis(carbazol-9-yl)benzene (mCP) [electron blocking layer] was then deposited on top of NPB layer at a rate of about 0.1 nm/s rate. Emitting compound EC-1 (6 wt %) was co-deposited as an emissive layer with host material Host-3 at about 0.01 nm/s and about 0.10 nm/s, respectively, to make the appropriate thickness ratio. 2,8-bis(diphenylphosphoryl)dibenzo[b,d]thiophene (PPT) [electron transporting material] was then deposited at about 0.1 nm/s rate on the emissive layer. A layer of lithium fluoride (LiF) (electron injection material) was deposited at about 0.005 nm/s rate followed by deposition of the cathode as Aluminum (Al) at about 0.3 nm/s rate. The representative device structure was: ITO (about 110 nm thick)/PEDOT:PSS (about 30 nm thick)/NPB (about 40 nm thick)/mCP (about 10 nm thick)/EC-1: Host-3 (about 20 nm thick)/PPT (about 40 nm thick)/LiF (about 0.8 nm thick)/Al (about 70 nm thick). The device was then immediately encapsulated with a glass cap to cover the emissive area of the OLED device in order to protect from moisture, oxidation or mechanical damage inside a glove box.

ITO/PEDOT (30 nm)/NPB (40 nm)/mCP (10 nm)/6 wt % EC-1 in hosts (20 nm)/PPT (40 nm)/LiF (0.8 nm)/Al (70 nm)

Hosts used:

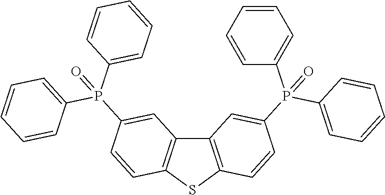 [SMILES: O=P(c1ccccc1)(c1ccccc1)c1ccc2sc3ccc(P(=O)(c4ccccc4)c4ccccc4)cc3c2c1]

dibenzo[b,d]thiophene-2,8-diylbis(diphenylphosphine oxide) (PPT [Host-1])

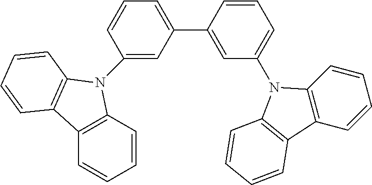 [SMILES: c1cc(-c2cccc(-n3c4ccccc4c4ccccc43)c2)cc(-n2c3ccccc3c3ccccc32)c1]

3,3′-di(9H-carbazol-9-yl)-1,1′-biphenyl (mCBP [Host-2])

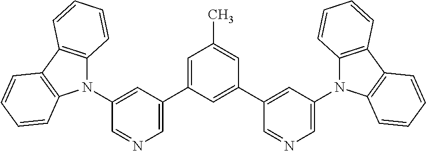 [SMILES: Cc1cc(-c2cncc(-n3c4ccccc4c4ccccc43)c2)cc(-c2cncc(-n3c4ccccc4c4ccccc43)c2)c1]

9,9′-(5,5′-(5-methyl-1,3-phenylene)bis(pyridine-5,3-diyl))bis(9H-carbazole) (Host 3)

### Emitting Compound (EC-1);

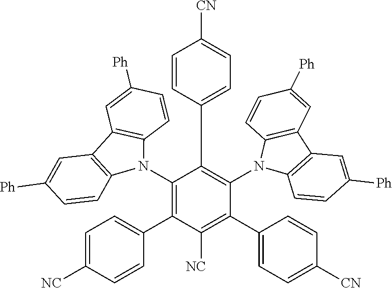 [SMILES: N#Cc1ccc(-c2c(C#N)c(-c3ccc(C#N)cc3)c(-n3c4ccc(-c5ccccc5)cc4c4cc(-c5ccccc5)ccc43)c(-c3ccc(C#N)cc3)c2-n2c3ccc(-c4ccccc4)cc3c3cc(-c4ccccc4)ccc32)cc1]

Device Characteristics;

| | Voltage at | | | | |
|---|---|---|---|---|---|
| Host | 1K | EL (nm) | PE [lm/W] | LE [cd/A] | EQE [%] |
| Host-1 | 9.44 | 491 | 10 | 31 | 12.8 |
| Host-2 | 8.94 | 491 | 11.8 | 33 | 13.7 |
| Host-3 | 6.24 | 491 | 23 | 47 | 18.5 |

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained. At the very least, and not as an attempt to limit the application of the doctrine of equivalents to the scope of the claims, each numerical parameter should at least be construed in light of the number of reported significant digits and by applying ordinary rounding techniques.

The terms “a,” “an,” “the” and similar referents used in the context of describing the invention (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claim. No language in the specification should be construed as indicating any non-claimed element essential to the practice of the invention.

Groupings of alternative elements or embodiments disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, the claims include all modifications and equivalents of the subject matter recited in the claims as permitted by applicable law. Moreover, any combination of the above-described elements in all possible variations thereof is contemplated unless otherwise indicated herein or otherwise clearly contradicted by context.

In closing, it is to be understood that the embodiments disclosed herein are illustrative of the principles of the claims. Other modifications that may be employed are within the scope of the claims. Thus, by way of example, but not of limitation, alternative embodiments may be utilized in accordance with the teachings herein. Accordingly, the claims are not limited to embodiments precisely as shown and described.

1. An emissive compound represented by Formula 1: 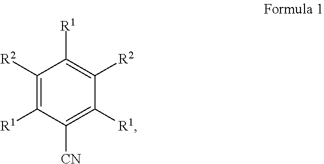
wherein each R¹ is independently an optionally substituted phenyl, optionally substituted pyridinyl, or optionally substituted pyrimidinyl; and each R² is independently an optionally substituted carbazolyl, an optionally substituted benzocarbazolyl, an optionally substituted phenoxazinyl, an optionally substituted phenothiazinyl, or an optionally substituted dihydrophenazinyl.

2. The compound of claim 1, wherein any substituent present has a molecular weight of 15 Daltons to 500 Daltons.

3. The compound of claim 2, wherein R¹ is optionally substituted phenyl.

4. The compound of claim 2, wherein R¹ is optionally substituted pyridin-3-yl.

5. The compound of claim 2, wherein R² is optionally substituted carbazolyl.

6. The compound of claim 2, wherein R² is optionally substituted benzocarbazolyl.

7. The compound of claim 1, wherein R¹ is:

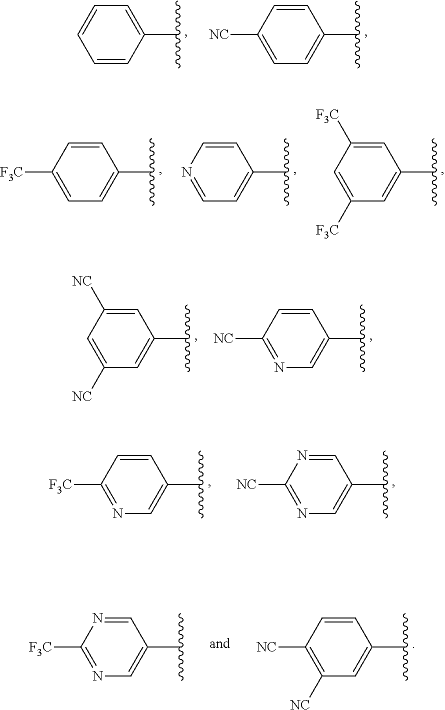

8. The compound of claim 1, wherein R² is: 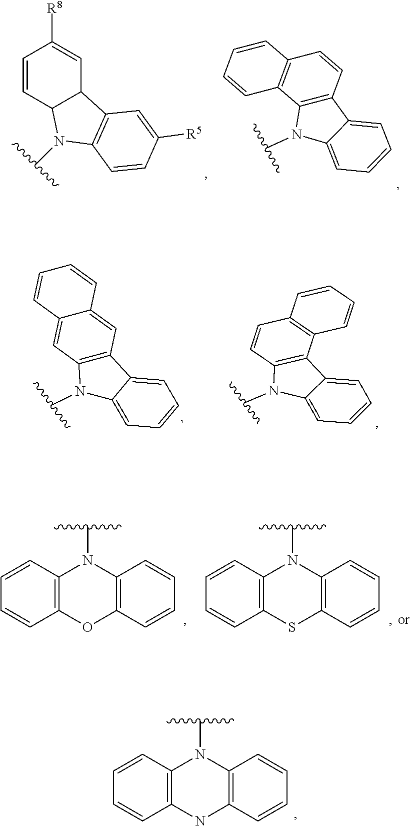
wherein each R⁸ and R⁵ are independently optionally substituted phenyl, optionally substituted amine, optionally substituted carbazolyl, optionally substituted phenoxazinyl, optionally substituted phenothiazinyl, optionally substituted dihydrophenazinyl, a C₁-C₃ alkyl or hydrogen.

9. The compound of claim 1, wherein the compound is:

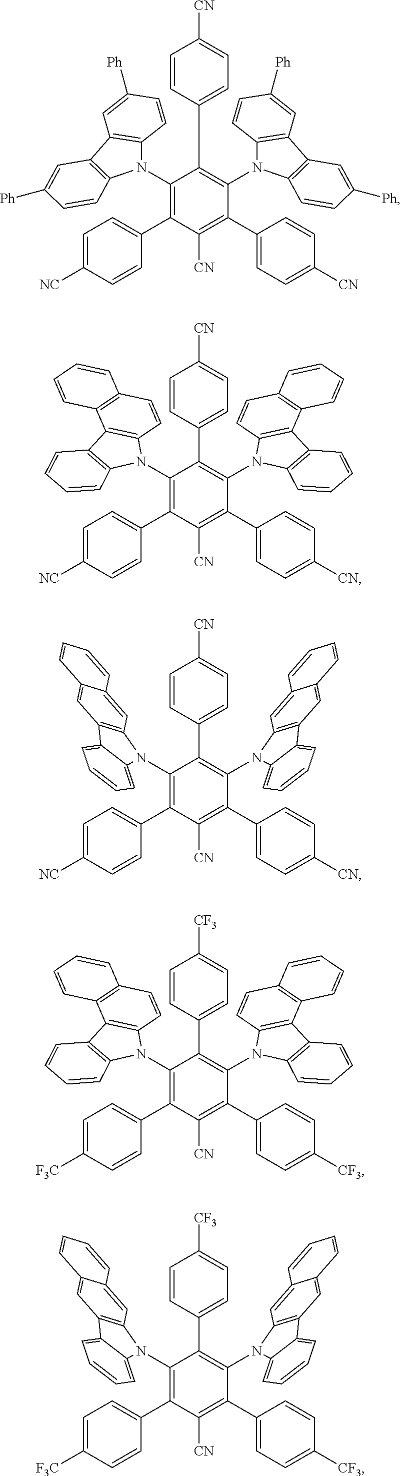

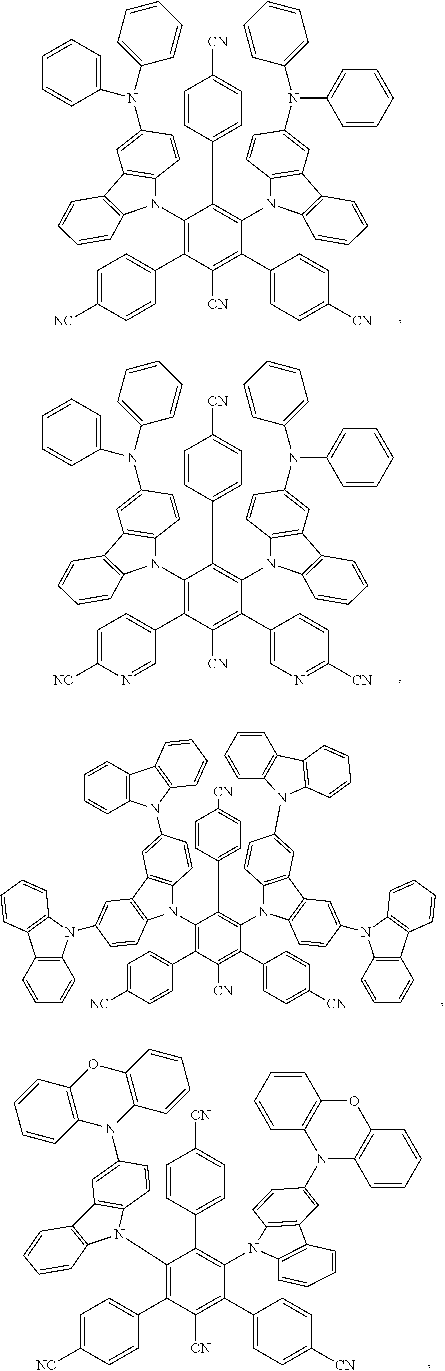

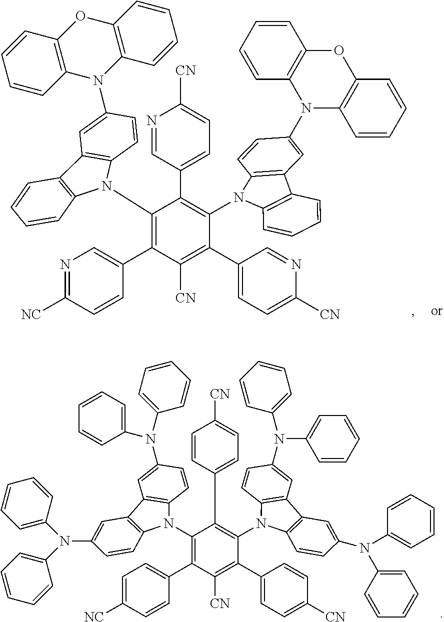 [SMILES: N#Cc1ccc(-c2c(C#N)c(-c3ccc(C#N)nc3)c(-n3c4ccccc4c4cc(N5c6ccccc6Oc6ccccc65)ccc43)c(-c3ccc(C#N)nc3)c2-n2c3ccccc3c3cc(N4c5ccccc5Oc5ccccc54)ccc32)cn1 ; N#Cc1ccc(-c2c(C#N)c(-c3ccc(C#N)cc3)c(-n3c4ccc(N(c5ccccc5)c5ccccc5)cc4c4cc(N(c5ccccc5)c5ccccc5)ccc43)c(-c3ccc(C#N)cc3)c2-n2c3ccc(N(c4ccccc4)c4ccccc4)cc3c3cc(N(c4ccccc4)c4ccccc4)ccc32)cc1]

10. The compound of claim 1, wherein the compound is fluorescent or phosphorescent.

11. The compound of claim 1, wherein the compound is electroluminescent.

12. A light-emitting element comprising the compound of claim 1.

13. A light-emitting device comprising the light-emitting element of claim 12.

14. An organic light-emitting diode device comprising:
a cathode;
an anode; and
a light-emitting layer disposed between and electrically connected to the anode and the cathode, wherein the light-emitting layer comprises an emitting compound according to claim 2.

15. The device of claim 14, further comprising a hole-transport layer between the anode and the light-emitting layer and an electron-transport layer between the cathode and the light-emitting layer.

16. The device of claim 14, wherein R¹ is optionally substituted phenyl.

17. The device of claim 14, wherein R¹ is optionally substituted pyridin-3-yl.

18. The device of claim 14, wherein R² is optionally substituted carbazolyl.

19. The device of claim 14, wherein R² is optionally substituted benzocarbazolyl.

20. A compound represented by the formula 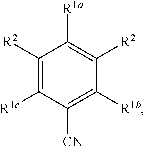
wherein R^{1a} is 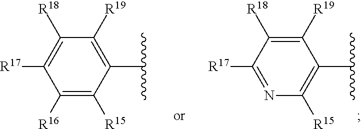
and
wherein R^{1b} is 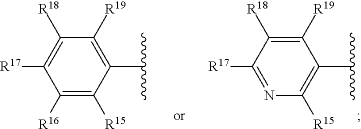
and.
wherein R^{1c} is 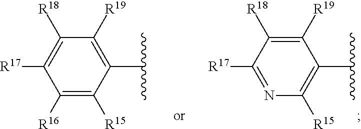
and
each R² is independently 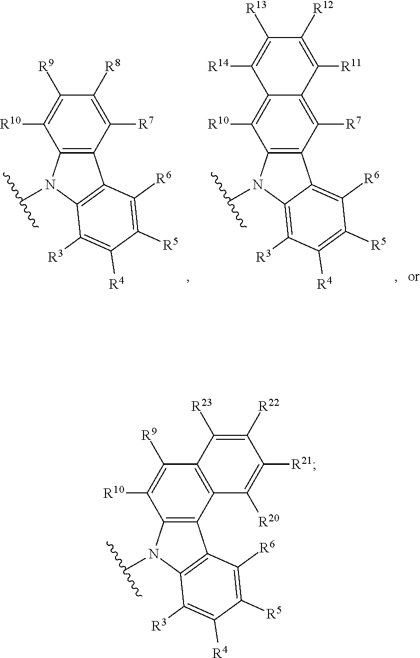
and
wherein R³, R⁴, R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², and R²³, are independently H or any substituent, such as a substituent having from 0 to 6 carbon atoms and from 0 to 5 heteroatoms, wherein each heteroatom is independently: O, N, P, S, F, Cl, Br, or I; and/or having a molecular weight of 15 g/mol to 300 g/mol.
